(19) United States
(12) Patent Application Publication
(10) Pub. No.: US 2025/0082600 A1
(43) Pub. Date: Mar. 13, 2025

(54) TREATING HAEMATOLOGICAL MALIGNANCIES BY MEANS OF SATRAPLATIN

(71) Applicant: PHARMA& SCHWEIZ GMBH, Cham (CH)

(72) Inventors: Felix DAHM, Zug (CH); Thilo Joachim ZANDER, Zug (CH); Christoph Robert RENNER, Zug (CH); Gabriel Benjamin MARKSON, Zug (CH)

(21) Appl. No.: 18/726,703

(22) PCT Filed: Dec. 20, 2022
(86) PCT No.: PCT/EP2022/087090

§ 371 Date: Jul. 3, 2024

(30) Foreign Application Priority Data
Jan. 7, 2022 (GB) 2200170.5

(51) Int. Cl. A61K 31/282; A61P 35/00; C12Q 1/6886

(52) U.S. Cl. CPC A61K 31/282; A61P 35/00; C12Q 1/6886; C12Q 2600/106; C12Q 2600/156; C12Q 2600/158

(57) ABSTRACT

The present invention relates to medical uses and methods of treatment employing satraplatin. These medical uses are beneficial in treatment of cancers, particularly haematological malignancies. The invention also relates to methods of patient stratification and methods for the selection of treatment regimens, as well as to diagnostic methods and to biomarker panels.

### FIELD OF THE INVENTION

The present invention relates to medical uses and methods of treatment employing satraplatin. These medical uses are beneficial in treatment of cancers, particularly haematological malignancies. The invention also relates to methods of patient stratification and methods for the selection of treatment regimens, as well as to diagnostic methods and to biomarker panels.

### BACKGROUND

Cisplatin, carboplatin and oxaliplatin are platinum-based drugs that are used throughout the world for cancer treatment (1). All three are intravenously administered and primarily hydrophilic with similar or even overlapping side-effects including nephrotoxicity, ototoxicity, neurotoxicity, cardiotoxicity, haematological toxicity, hepatotoxicity, and gastrointestinal toxicity. Platinum toxicity is usually the main reason for dose adaptations or even treatment suspension. This is particularly true for cisplatin with the two most common nephrotoxic side effects including acute kidney injury and hypomagnesemia, which is reported to affect up to 90% of cisplatin treated patients (2). Neurotoxicity and ototoxicity (60-90% of all patients) are the second and third most common platinum toxicities with oxaliplatin causing primarily neurotoxicity and cisplatin causing ototoxicity. In order to develop better tolerated platinum compounds with less toxicity, platinum (IV) complexes featuring an octahedral geometry with two additional ligand sites were developed. They follow a classical prodrug-concept since it is essential for their anticancer activity that they are reduced to the corresponding platinum(II) analogues in the tumour cell leading to apoptotic cell (3). Satraplatin (bis-acetato-ammine-dichloro-cyclohexylamine-platinum) is the prime example for a novel platinum(IV) compound and has been developed as the first oral fourth generation platinum compound with activity in platinum-sensitive and even some platinum-resistant preclinical models (4). Over the last two decades, satraplatin was studied extensively in different in-vitro and in-vivo solid tumour models (5) and demonstrated consistently high activity (6).

Clinical development was done in a variety of cancer entities including prostate cancer with encouraging results, although prostate cancer was traditionally considered a platinum-resistant disease. However, phase I/II satraplatin monotherapy trials in hormone refractory prostate cancer patients (HRPC) had demonstrated an objective response rate of up to 31% (7). On the basis of these encouraging data, a large phase Ill registration trial (SPARC) was initiated evaluating satraplatin as second-line therapy in combination with prednisone versus prednisone and placebo for HRPC patients who progressed after one line of chemotherapy. The trial met its primary endpoint and showed a 33% reduction in risk of progression or death (PFS; HR: 0.67; p<0.001), a 36% reduction in the risk of pain progression, PSA responses (PSA decline of at least 50%) of 25% vs 12% (p=0.001) and an objective RECIST response rate of 8% vs 0.7% (p<0.002). Unfortunately, no statistically significant overall survival (OS) benefit (14.3 months vs 14.3 months; HR: 0.98; p=0.80) was noted and the clinical development stopped (8). As a consequence, satraplatin is not approved for cancer treatment.

In agreement with data from previous clinical trials, the SPARC trial confirmed that satraplatin related toxicity was generally mild including moderate nausea, fatigue, anorexia, diarrhoea, and altered taste. In particular, there was no nephrotoxicity, hair loss or added neurotoxicity noted which renders this drug unique within this class of drugs.

There is a clear clinical need for novel biomarkers that can be used to identify patients that would benefit from treatment with satraplatin in view of the drug's advantageous properties, including high activity and reduced toxicity.

### SUMMARY OF THE INVENTION

The invention provides a number of aspects that relate to medical uses.

In a first aspect, the invention provides satraplatin for use in the treatment of a haematological malignancy in a patient identified as likely to benefit from such treatment by analysis of a gene selected from the group consisting of: a gene located in the 9p21 locus (such as MTAP, CDKN2B, CDKN2A, or DMRTA1); BCL2; TULP3; AOC1; BOC1; DDX3X; CRYBG3; SLFN5; ETNK2; BRCA2; USP22; KIAA1683; AP4E1; PAN3; AC073343.11.1; PHF2; KLHDC1; and WRN.

In a second aspect, the invention provides a method of treating a haematological malignancy, the method comprising administering satraplatin to a patient identified as likely to benefit from such treatment on the basis of analysis of a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN.

In a third aspect, the invention provides a method of treating a haematological malignancy, the method comprising:
obtaining a sample comprising a cell of the haematological malignancy;
analysing the cell of the haematological malignancy for a change associated with a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; and
administering satraplatin to a patient identified as likely to benefit from such treatment on the basis of the analysis.

In a fourth aspect, the invention provides satraplatin for use in the treatment of a haematological malignancy associated with a loss of the 9p21 locus.

In a fifth aspect, the invention provides satraplatin for use in the treatment of a haematological malignancy associated with a deficiency of a gene selected from the group consisting of: MTAP; CDKN2B; CDKN2A; and DMRTA1. The deficiency may be a loss of copy number in respect of the gene.

In a sixth aspect, the invention provides satraplatin for use in the treatment of a haematological malignancy associated with mutation of a gene selected from the group consisting of: BCL2; AOC1; BOC1; DDX3X; CRYBG3; SLFN5; ETNK2; BRCA2; USP22; KIAA1683; and AP4E1.

In a seventh aspect, the invention provides satraplatin for use in the treatment of a haematological malignancy with elevated expression of TULP3; PAN3; AC073343.11.1; PHF2; KLHDC1; BRCA2; or WRN.

In a suitable embodiment of any of the first to seventh aspects of the invention, the haematological malignancy may be selected from the group consisting of: a lymphoma; a myeloma; and a leukaemia. Particular forms of such haematological malignancies are considered elsewhere in the specification.

In an eighth aspect, the invention provides satraplatin for use as a second line therapy in the treatment of cancer non-responsive to immune checkpoint therapy.

In a ninth aspect, the invention provides a method of treating a cancer that is non-responsive to immune checkpoint therapy, the method comprising administering satraplatin to a patient with such a cancer.

The invention also provides a number of aspects that relate to patient stratification/selection of treatment regimens.

In a tenth aspect, the invention provides a method of determining the likelihood of a haematological malignancy responding favourably to satraplatin treatment, the method comprising:
analysing a cell of the haematological malignancy for a change associated with a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; wherein
identification of a change in the gene analysed indicates that the haematological malignancy is likely to respond favourably to satraplatin treatment.

The invention also provides a number of aspects that relate to methods of diagnosis.

In a eleventh aspect, the invention provides a method comprising analysing at least one gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; in a cell of a haematological malignancy from a patient, wherein
when the gene is a gene located in the 9p21 locus, the analysis comprises determining if a deficiency is present, for example by assessing copy number;
when the gene is selected from the group consisting of: BCL2; AOC1; BOC; DDX3X; CRYBG3; SLFN5; ETNK2; BRCA2; USP22; KIAA1683; and AP4E1, the analysis comprises assaying for a mutation of the gene; and
when the gene is selected from the group consisting of: TULP3; PAN3; AC073343.11.1; PHF2; KLHDC1; BRCA2; and WRN, the analysis comprises assaying for elevated expression of the gene.

In a twelfth aspect, the invention provides a method comprising analysing a cell of a haematological malignancy from a patient for the presence of at least one gene mutation selected from the group set out in Table 1 and Table 2.

The invention also provides a number of aspects that relate to biomarker panels.

In a thirteenth aspect, the invention provides a signature panel characteristic of sensitivity of a haematological malignancy to treatment with satraplatin, the panel comprising:
loss of copy number of at least one gene selected from the group consisting of: MTAP; CDKN2B; CDKN2A; and DMRTA1; and/or
a mutation of at least one gene selected from the group consisting of: BCL2; AOC1; BOC; DDX3X; CRYBG3; SLFN5; ETNK2; BRCA2; USP22; KIAA1683; and AP4E1; and/or
elevated expression of at least one gene selected from the group consisting of: TULP3; PAN3; AC073343.11.1; PHF2; KLHDC1; BRCA2; and WRN.

In a fourteenth aspect, the invention provides a signature biomarker panel characteristic of sensitivity of a haematological malignancy to treatment with satraplatin, the panel comprising at least one gene mutation selected from the group set out in Table 1 and Table 2.

In a suitable embodiment of any of the first to seventh, tenth, or eleventh to fourteenth aspects of the invention, the haematological malignancy may be selected from the group consisting of: a lymphoma; a myeloma; and a leukaemia. Specific forms of such haematological malignancies are considered elsewhere in the specification.

The genetic mutation spectrum of cell lines and thus tumour diseases is highly diverse and numerous. Predicting the presence of a mutation/deletion/methylation as a biomarker for heightened response and thus increased efficacy is impossible a priori. This requires an integrated and complex analysis such as the one described herein in the context of hematological malignancies.

In addition, data are inconsistent regarding the prognostic significance of MTAP. Studies have been published showing that MTAP loss leads to a worse prognosis in patients receiving chemotherapy in a number of cancers (NSCLC, Mantle Cell Lymphoma, Ewing Sarcoma). Other authors have performed gene analysis to find relevant biomarkers in order to predict treatment responses in cancer. As an example, an investigation in ovarian carcinoma can be mentioned, where MTAP was considered as a relevant biomarker and therefore analysed. However, there was no association between MTAP deletion with response to therapy.

The present invention is not obvious, at least because, given the complex data situation outlined above, it would not be possible for the skilled person to make a priori predictions of relevant biomarkers for the optimized efficacy of satraplatin.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows cytotoxic activity of satraplatin in tumour cell lines of different origin as determined by AUC values as described. The median AUC value across all tested cell lines is 2.35. Tumour entities are indicated on the x-axis. There is a significant higher activity in haematopoietic & lymphoid malignancies when compared solid organ cancers (ANOVA p-value=2.3e-08).

FIG. 2 shows enhanced killing of haemato-oncologic cell lines using satraplatin versus cisplatin. Depicted is the delta of IC50 (μM) values for cisplatin minus satraplatin.

FIG. 3 shows a volcano plot of gene expression—AUC correlation analysis across hematopoietic and lymphoid cancers. Genes conferring enhanced satraplatin sensitivity are depicted on the left, those conferring resistance on the right. Genes achieving statistical significance are marked as black squares (sensitive) and black triangles (resistant).

FIG. 4 shows a volcano plot of signature genes dissecting more sensitive and less sensitive groups in hematopoietic and lymphoid cancers on mutation level. Gene mutations conferring enhanced satraplatin sensitivity are depicted on the left, those conferring resistance on the right. Genes achieving statistical significance are marked as black squares (sensitive) and black triangles (resistant).

FIG. 5 shows a volcano plot of signature genes dissecting more sensitive (left) and less sensitive (right) groups in hematopoietic and lymphoid cancers on DNA copy number deletion level. Genes achieving statistical significance are marked as black squares (sensitive).

FIG. 6 shows correlation of satraplatin IC50 activity in μM (x-axis) versus the list of examined cell lines (y-axis). Cell lines are listed according their satraplatin sensitivity with the most sensitive ones listed at the top. MTAP status of the individual cell lines is coded as follows: Grey/Gray=MTAP wt; Black=MTAP deficient; White=status undefined/unknown.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION OF THE INVENTION

The invention is based, at least in part, on the inventors' identification of a number of gene-associated changes which, when present in cells of a haematological malignancy, are indicative of the sensitivity of that cancer to the chemotherapeutic agent satraplatin.

The identification of markers by which blood cancers sensitive to satraplatin can be identified offers significant advantages in terms of allowing treatment with satraplatin to be targeted to those patients that will gain therapeutic advantage from the treatment. It is well recognised that the ability to target treatments to suitable patients is important in both achieving optimal clinical outcome (by virtue of ensuring that a patient is provided with a treatment with a high probability of efficacy from the earliest time of treatment) and avoiding unnecessary or ineffective treatment (which can unduly delay successful treatment, while causing unneeded side-effects) using therapeutic agents that will not achieve the desired outcomes.

While the genetic changes in question are described more fully below, certain key changes noted by the inventors include deficiencies, such as loss of copy number, in respect of genes of the 9p21 locus (in particular MTAP, but also CDKN2B, CDKN2A and DMRTA1), mutation of a sub-set of genes (exemplified by, but not limited to, BCL2), and elevated expression of a panel of proteins including TULP3. Each of the changes identified, when present in a cell of a haematological malignancy, indicates that the cancer will be susceptible to successful treatment with satraplatin.

The finding that cancers with loss of locus 9p21 (which contains MTAP, CDKN2B, CDKN2A and DMRTA1) are made more sensitive to treatment with satraplatin identifies this agent as very useful in treatment of these cancers. Loss of 9p21 confers resistance to immune checkpoint therapies that may otherwise be used in the treatment of such cancers, so satraplatin thus provides a needed second line therapy for cancers that may otherwise prove difficult to treat.

From the foregoing, it will be appreciated that the inventors' findings enable new methods of treatment and medical uses of satraplatin, and also new methods by which patients can be stratified and appropriate methods of cancer treatment selected. The findings also make available new methods useful in the diagnosis of cancers (and particularly haematological malignancies), as well as new and useful panels of biomarkers. These different aspects of the invention are described in more detail under the appropriate headings below.

### Methods of Treatment and Medical Uses of the Invention

In its first to ninth aspects, the invention relates to methods of treatment using satraplatin, or to medical uses of satraplatin. The methods of treatment and medical uses defined relate to the use of satraplatin in treatment of specific forms of cancer.

In particular, the first to seventh aspects of the invention relate to methods of treatment and medical uses of utility in the treatment of haematological malignancies that can be characterised with reference to genetic changes or protein over-expression. The eighth and ninth aspects of the invention are directed to methods of treatment and medical uses to be employed in the treatment of cancers characterised with reference to their resistance to other forms of cancer therapy (particularly their lack of response to immune checkpoint therapies).

These aspects of the invention are based, to at least some extent, upon the inventors' identification of a number of genetic changes that increase or decrease the sensitivity of cancer cells, such as those of haematological malignancies, to satraplatin, and thus indicate that patients with cancers having such genetic changes will, or will not, benefit from treatment with this agent. The changes identified are discussed in more detail elsewhere.

The inventors are the first to recognise that identifying whether or not these changes are present in a haematological malignancy provides valuable guidance as to how the haematological malignancy in question may be treated effectively.

In the first and second aspects of the invention analysis of a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN allows identification of a patient with a haematological malignancy that is likely to benefit from cancer treatment using satraplatin. The analysis may identify a change associated with one or more of the genes that is indicative of increased sensitivity to satraplatin, suggesting that the haematological malignancy will respond well to this chemotherapeutic agent.

In the methods of the third aspect of the invention, a cell from a patient's haematological malignancy is obtained in a sample, and the sample analysed with respect to a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN.

Again, in the case that a change associated with one or more of the genes is identified, this suggests that the haematological malignancy will respond well to satraplatin. Satraplatin is then administered to patients who have been identified in this manner as likely to benefit from such treatment.

In a suitable embodiment of any of the first, second or third aspects of the invention, the gene is one located in the 9p21 locus, and the analysis identifies a deficiency (such as reduced copy number) in respect of the gene. For example, the gene may be selected from the group consisting of: MTAP; CDKN2B; CDKN2A; and DMRTA1, and the analysis may identify a deficiency such as reduced copy numbers of the gene. In such embodiments, and particularly those in which reduced copy number is found to be present, the haematological malignancy may suitably be selected from the group consisting of: Burkitt lymphoma; DLBCL; CTCL; and multiple myeloma.

In a suitable embodiment of any of the first, second or third aspects of the invention, the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene. Specific examples of the mutations for which analysis may be conducted are considered elsewhere in the specification. In these embodiments, the haematological malignancy may suitably be selected from the group consisting of: Burkitt lymphoma; DLBCL; and ALCL ALK+.

In a further suitable embodiment of any of the first, second or third aspects of the invention, the gene is selected from the group consisting of: TULP3; PAN3; AC073343.11.1; PHF2; KLHDC1; BRCA2; and WRN, and the analysis identifies elevated expression of the gene.

The fourth, fifth, sixth and seventh aspects of the invention relate to medical uses of satraplatin in sub-populations of patients with haematological malignancies. In the case of the fourth aspect, the sub-population is one in which the haematological malignancy is associated with a loss of the 9p21 locus. In the fifth aspect, the sub-population is one in which the haematological malignancy is associated with a deficiency in respect of a gene selected from the group consisting of: MTAP; CDKN2B; CDKN2A; and DMRTA1 (such as reduced copy number of one or more genes selected from the group consisting of: MTAP; CDKN2B; CDKN2A; and DMRTA1). In the sixth aspect, the sub-population is one in which the haematological malignancy is associated with mutation of a gene selected from the group consisting of: BCL2; AOC1; BOC; DDX3X; CRYBG3; SLFN5; ETNK2; BRCA2; USP22; KIAA1683; and AP4E1. In the seventh aspect, the sub-population is one in which the haematological malignancy is associated with elevated expression of TULP3; PAN3; AC073343.11.1; PHF2; KLHDC1; BRCA2; or WRN. Each of these sub-populations represent newly identified groups of patients able to gain particular benefit from treatment of their haematological malignancy using satraplatin.

The eighth and ninth aspects of the invention are directed to methods of treatment and medical uses in which satraplatin is used as a second line therapy in the treatment of cancers that are non-responsive to immune checkpoint therapies.

The cancers in question may have become non-responsive to immune checkpoint therapies, or may never have been responsive to immune checkpoint therapies.

The cancer for which satraplatin is being used as a second line therapy may previously have been treated with an anti-PD-1 and/or an anti-PD-L1 therapeutic agent. For example, the cancer may previously have been treated with pembrolizumab.

It is known that loss of all or part of the 9p21 locus can alter the immune response within the tumour microenvironment in such a manner that immune checkpoint therapies become ineffective (Han et al., Nat Commun. 2021 Sep. 23; 12(1):5606). This aspect of the invention is based upon the inventors' new observation that such changes also heighten the cancer cells' sensitivity to satraplatin. Accordingly, satraplatin may be useful in treating cancers that fail to respond to immune checkpoint therapies, offering patients an important opportunity for further treatment of these cancers that may otherwise prove difficult to manage clinically. The skilled person will be able to identify methods by which it is possible to determine that a cancer is non-responsive to immune checkpoint therapy, including the use of suitable in vitro assays.

Suitably, a cancer that may benefit from treatment in accordance with the eighth or ninth aspect of the invention may be a solid tumour, such as those considered elsewhere in the present disclosure. Alternatively, the cancer may be a haematological malignancy.

### Patient Stratification and Selection of Treatment Regimens

The tenth aspect of the invention relates to methods that may be used in determining a suitable course of treatment for use in the management of a haematological malignancy. In particular, this aspect of the invention provides a method that allows a determination to be made as to whether a haematological malignancy is likely to respond favourably to satraplatin.

The method of the tenth aspect of the invention makes use again of the inventors' identification of changes associated with genes selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN that allow a prediction to be made as to whether cells of a haematological malignancy are sensitive to satraplatin. In the case of this aspect of the invention, this information is used to determine that haematological malignancies in which such changes are found to be present are likely to respond well to treatment with satraplatin.

In a suitable embodiment of the tenth aspect of the invention, the gene is one located in the 9p21 locus, and the analysis determines if a deficiency is present in respect of the gene. For example, the gene may be selected from the group consisting of: MTAP; CDKN2B; CDKN2A; and DMRTA1, and the analysis determines whether there is a deficiency of one or more of these genes.

In a suitable embodiment of the tenth aspect of the invention, the gene is one located in the 9p21 locus, and the analysis identifies reduced copy numbers of the gene. For example, the gene may be selected from the group consisting of: MTAP; CDKN2B; CDKN2A; and DMRTA1, and the analysis identifies reduced copy numbers of the gene. In such embodiments, the haematological malignancy may suitably be selected from the group consisting of: Burkitt lymphoma; DLBCL; CTCL; multiple myeloma; ALCL ALK+; and mantle cell lymphoma.

In a suitable embodiment of the tenth aspect of the invention, the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene. Specific examples of the mutations for which analysis may be conducted are considered elsewhere in the specification. In these embodiments, the haematological malignancy may suitably be selected from the group consisting of: Burkitt lymphoma; DLBCL; and ALCL ALK+.

In a further suitable embodiment of this aspect of the invention, the gene is selected from the group consisting of: TULP3; PAN3; AC073343.11.1; PHF2; KLHDC1; BRCA2; and WRN, and the analysis identifies elevated expression of the gene.

The methods described above are intended to allow identification of treatments that will kill cells of the haematological malignancy, thereby providing treatment of the disease.

Methods in accordance with the tenth aspect of the invention may include a step of selecting an appropriate course of treatment for a patient. Such methods may include a step of prescribing treatment for the patient, based upon the outcome of the method.

Methods in accordance with the tenth aspect of the invention may, in suitable embodiments, also comprise a further step of providing treatment using an agent identified as suitable. Thus a method of the tenth aspect of the invention may optionally comprise a further step of providing treatment with satraplatin, depending upon the results arrived at.

### Methods Useful in Diagnosis of Haematological Malignancies

Both the eleventh and twelfth aspects of the invention provide methods in which a cell of a haematological malignancy is analysed. These methods may be useful to clinicians or other parties providing care for patients with haematological malignancies.

In the methods of the eleventh aspect, the analysis is carried out in respect of at least one gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN. The analysis may be conducted to identify a change associated with said at least one gene.

When the gene is a gene located in the 9p21 locus (such as MTAP, CDKN2B, CDKN2A, or DMRTA1), the analysis may comprise determining if a deficiency is present in respect of the selected gene or genes. Deficiencies in the context of the present invention, and methods by which they may be analysed, are considered elsewhere in this disclosure.

By way of example, when the gene is a gene located in the 9p21 locus (such as MTAP, CDKN2B, CDKN2A, or DMRTA1), the analysis may comprise assessing copy number, and may involve identifying a change in copy number, if present. For the purposes of the present invention, a loss of copy number may be taken as constituting a deficiency in respect of the gene in question. In such an embodiment, the haematological malignancy may be selected from the group consisting of: Burkitt lymphoma; DLBCL; CTCL; multiple myeloma; ALCL ALK+; and mantle cell lymphoma.

When the gene is selected from the group consisting of: BCL2; AOC1; BOC; DDX3X; CRYBG3; SLFN5; ETNK2; BRCA2; USP22; KIAA1683; and AP4E1, the analysis comprises assaying for a mutation of the gene. Specific mutations of these genes are considered elsewhere in this specification, and one or more of these genes may be analysed to determine the presence or absence of one or more of the specific mutations disclosed in practicing methods in accordance with this aspect of the invention. In embodiments of the invention of this sort, the haematological malignancy may be selected from the group consisting of: Burkitt lymphoma; DLBCL; and ALCL ALK+.

When the gene is selected from the group consisting of: TULP3; PAN3; AC073343.11.1; PHF2; KLHDC1; BRCA2; and WRN, the analysis comprises assaying for elevated expression of the gene. Methods that may be used in carrying out such analysis are discussed elsewhere in the present disclosure.

In the case that the analysis is “positive” (which is to say that the analysis indicates the presence of a deficiency (such as a change in copy number, reduced gene expression, or no gene expression), the presence of a mutation, or the presence of elevated expression, as appropriate), this may indicate that the haematological malignancy in question will benefit from treatment with satraplatin.

The methods of the eleventh and twelfth aspects of the invention may optionally further comprise a step of obtaining a cell of the haematologic malignancy to be tested. The cell may be obtained as part of a blood sample, or by any other suitable means known to the skilled person.

### Biomarker Panels of the Present Invention

The thirteenth and fourteenth aspects of the invention provide signature biomarker panels, the presence of which in a sample of a haematological malignancy allows the malignancy to be characterised as sensitive to treatment with satraplatin.

The biomarker panel of the thirteenth aspect of the invention is defined with reference to a number of gene-associated changes indicative of sensitivity to satraplatin treatment. Such changes may include loss of copy number of at least one gene selected from the group consisting of: MTAP; CDKN2B; CDKN2A; and DMRTA1. Alternatively, or additionally, such changes may include a mutation of at least one gene selected from the group consisting of: BCL2; AOC1; BOC; DDX3X; CRYBG3; SLFN5; ETNK2; BRCA2; USP22; KIAA1683; and AP4E1. The changes may also, or as an alternative, include elevated expression of at least one gene selected from the group consisting of: TULP3; PAN3; AC073343.11.1; PHF2; KLHDC1; BRCA2; and WRN.

The biomarker panel of the fourteenth aspect of the invention is defined with reference to the presence of one or more of a number of genetic mutations indicative of sensitivity of a haematological malignancy to treatment with satraplatin, these mutations being set out in Tables 1 and 2.

The signature biomarker panels of the invention provide newly identified combinations of biomarkers that are useful in the characterisation of haematological conditions in a manner that will facilitate their treatment. These combinations, and their relevance in determining sensitivity of a haematological condition to treatment with satraplatin, have not previously been recognised or reported.

A biomarker panel in accordance with either the thirteenth or fourteenth aspect of the invention comprises at least one of the markers recited in Tables 1 and 2, and may comprise all of the markers recited in Tables 1 and 2.

Relevant biomarkers may suitably be determined based on a diagnosis of a haematological malignancy of interest (which may be performed using traditional methods, such as via histological or genetic markers), considered in combination with the disclosure of the present invention.

A biomarker panel of the invention may comprise all of the markers recited in Tables 1 and 2 in respect of a haematological malignancy of interest, or may comprise a subset of such markers.

Merely by way of example, a biomarker panel for use in respect of the haematological malignancy DLBCL NOS (GCB) may comprise:
one or more mutations in a gene selected from the group consisting of: BCL2; AOC1; BOC; DDX3X; CRYBG3; ETNK2; SLFN5; BRCA2; FRG1B; KIAA1683; USP22; and AP4E1 and/or
a loss of copy number, or reduced copy number, in respect of one or more genes selected from the group consisting of: MTAP; CDKN2B; and CDKN2A.

A biomarker panel in accordance with this embodiment of the invention may comprise one or more, two or more, three or more, four or more, five or more, six or more, seven or more, eight or more, nine or more, ten or more, or eleven or more of the mutations referred to above. Suitably, such a biomarker panel may comprise all the mutations referred to above.

Alternatively, or additionally, a biomarker panel in accordance with this embodiment of the invention may comprise one or more, or two or more of the losses of copy number referred to above. Suitably, such a biomarker panel may comprise all of the losses of copy number referred to above.

A biomarker panel for use in respect of the haematological malignancy DLBCL NOS (ABC) may comprise:
one or more mutations in a gene selected from the group consisting of: BCL2; and DDX3X.

A biomarker panel in accordance with this embodiment of the invention may comprise one or both of the mutations referred to above.

A biomarker panel for use in respect of the haematological malignancy Burkitt lymphoma may comprise:
one or more mutations in a gene selected from the group consisting of: BCL2; AOC1; BOC; DDX3X; BRCA2; FRG1B; USP22; and AP4E1 and/or
a loss of copy number, or reduced copy number, in respect of each of MTAP, CDKN2B, CDKN2A and DMRTA1.

A biomarker panel in accordance with this embodiment of the invention may comprise one or more, two or more, three or more, four or more, five or more, six or more, or seven or more of the mutations referred to above. Suitably, such a biomarker panel may comprise all the mutations referred to above.

Alternatively, or additionally, a biomarker panel in accordance with this embodiment of the invention may comprise one or more, two or more, or three or more of the losses of copy number referred to above. Suitably, such a biomarker panel may comprise all of the losses of copy number referred to above.

A biomarker panel for use in respect of the haematological malignancy ALCL ALK+ may comprise:
one or more mutations in a gene selected from the group consisting of: DDX3X; SLFN5; and AP4E1 and/or
a loss of copy number, or reduced copy number, in respect of each of MTAP, CDKN2B, CDKN2A and DMRTA1.

A biomarker panel in accordance with this embodiment of the invention may comprise one or more, or two or more of the mutations referred to above. Suitably, such a biomarker panel may comprise all the mutations referred to above.

Alternatively, or additionally, a biomarker panel in accordance with this embodiment of the invention may comprise one or more, two or more, or three or more of the losses of copy number referred to above. Suitably, such a biomarker panel may comprise all of the losses of copy number referred to above.

A biomarker panel for use in respect of the haematological malignancy multiple myeloma may comprise:
one or more mutations in a gene selected from the group consisting of: BRCA2; and FRG1B and/or
a loss of copy number, or reduced copy number, in respect of one or more genes selected from the group consisting of: MTAP; CDKN2B; and CDKN2A.

A biomarker panel in accordance with this embodiment of the invention may comprise one or both of the mutations referred to above.

Alternatively, or additionally, a biomarker panel in accordance with this embodiment of the invention may comprise one or more, or two or more of the losses of copy number referred to above. Suitably, such a biomarker panel may comprise all of the losses of copy number referred to above.

A biomarker panel for use in respect of the haematological malignancy mantle cell lymphoma may comprise:
one or more mutations in FRG1B and/or
a loss of copy number, or reduced copy number, in respect of each of MTAP, CDKN2B, CDKN2A and DMRTA1.

A biomarker panel in accordance with this embodiment of the invention may comprise one or more, two or more, or three or more of the losses of copy number referred to above. Suitably, such a biomarker panel may comprise all of the losses of copy number referred to above.

Alternatively, or additionally, such a biomarker panel may also comprise the mutations set out above.

A biomarker panel for use in respect of the haematological malignancy CTCL may comprise:
a loss of copy number, or reduced copy number, in respect of each of MTAP, CDKN2B, CDKN2A and DMRTA1.

A biomarker panel in accordance with this embodiment of the invention may comprise one or more, two or more, or three or more of the losses of copy number referred to above. Suitably, such a biomarker panel may comprise all of the losses of copy number referred to above.

A cell of a haematological malignancy may include one or more markers of a biomarker panel in accordance with the invention. The presence of such markers may allow the haematological malignancy to be characterised as sensitive to treatment with satraplatin. Alternatively, in the event that a cell of a haematological malignancy does not comprise one or more markers of a biomarker panel in accordance with the invention, this may allow the haematological malignancy to be characterised as insensitive to treatment with satraplatin. The considerations set out above with respect to biomarker panels relevant to specific recited haematological malignancies are also applicable to the biomarkers that may be present in cells representative of such malignancies.

Changes Associated with Genes or a Locus of Interest: Mutation, Elevated Expression, and Deficiency of Genes, and Loss of Locus

The inventors have identified a number of gene-associated changes that, when present within cells of a haematological malignancy, provide useful guidance as to suitable treatment regimens that may be used to treat the haematological malignancy. These changes include mutation of a gene or genes, elevated expression of a gene or genes, and a deficiency of a gene or genes. Each of these is defined further below.

The gene-associated changes may be investigated singly or in independently selected combinations in respect of a gene or genes of interest.

The inventors have also identified that loss of a particular locus is associated with a beneficial change in sensitivity of a haematological malignancy to satraplatin. This loss may be investigated in combination with one or more of the gene-associated changes referred to herein.

Specific details of exemplary changes identified by the inventors are set out below, and the considerations in the following paragraphs should be used, except for where the context requires otherwise, when interpreting the relevant changes associated with these genes in the various aspects and embodiments of the invention.

### Loss of the 9p21 Locus or Associated Genes

The inventors have found loss of the 9p21 locus in cells of a haematological malignancy to be associated with an increase in sensitivity of the malignancy to satraplatin. Accordingly, loss of the 9p21 locus, or of genes associated with this locus, may be used as an indication that a haematological malignancy will respond well to treatment with satraplatin.

### Mutation

The inventors have identified that mutations in one or more of the genes BCL2; AOC1; BOC; DDX3X; CRYBG3; SLFN5; ETNK2; BRCA2; USP22; KIAA1683; and AP4E1 present in the cells of a haematological malignancy indicates that these cells have increased sensitivity to satraplatin, and that this compound may thus be used effectively in treatment of the haematological malignancy.

A mutation in respect of any of these genes of interest may be any change in the gene present in a cell of a haematological malignancy as compared to the wild type gene. Such a change may not cause a change in the protein gene product (a “silent” mutation), or may cause a detectable alteration in the gene product. Analysis of a gene mutation may be practiced by determining the presence of changes in the nucleic acid sequence of the gene or gene transcript, or by determining the presence of changes in the gene product (which, for the present purposes, may also include determining the absence of the gene product).

By way of example, a mutation may comprise one or more of the following changes in the nucleic acid sequence of a gene or gene transcript as compared to the wild type gene (or its transcripts): a deletion; an insertion; a substitution; or a truncation (particularly in respect of a gene transcript). Such mutations may result in changes to the gene transcript or gene product, or may alter transcription or translation processes, such as altering splicing of the gene transcript (e.g. to generate splice acceptor variants) such that alternative gene products are generated.

Similarly, a mutation may comprise one or more of the following changes in the amino acid sequence of a gene product, as compared to sequence of the product of the wild type gene): a deletion; an insertion; a substitution; a truncation; or absence of the gene product.

Mutations may be analysed in respect of one or more genes independently selected from the group consisting of: BCL2; AOC1; BOC; DDX3X; CRYBG3; SLFN5; ETNK2; BRCA2; USP22; KIAA1683; and AP4E1. Suitably a mutation analysed is in respect of BCL2 and/or AOC1 and/or BOC and/or DDX3X and/or CRYBG3 and/or SLFN5 and/or ETNK2 and/or BRCA2 and/or USP22 and/or KIAA1683 and/or AP4E1.

In a suitable embodiment, a mutation is in respect of BCL2. In a suitable embodiment, a mutation is in respect of AOC1. In a suitable embodiment, a mutation is in respect of BOC. In a suitable embodiment, a mutation is in respect of DDX3X. In a suitable embodiment, a mutation is in respect of CRYBG3. In a suitable embodiment, a mutation is in respect of SLFN5. In a suitable embodiment, a mutation is in respect of ETNK2. In a suitable embodiment, a mutation is in respect of BRCA2. In a suitable embodiment, a mutation is in respect of USP22. In a suitable embodiment, a mutation is in respect of KIAA1683. In a suitable embodiment, a mutation is in respect of AP4E1. In each of these cases a mutation may be analysed in respect of only the gene identified, or in respect of the gene identified in combination with one or more other genes. In each of these cases a mutation may be present in respect of only the gene identified, or in respect of the gene identified in combination with one or more other genes.

Details of specific mutations that the inventors have identified as being of particular relevance to satraplatin sensitivity in named cell lines, and hence in the haematological malignancies of which these cell lines are representative, are set out in Tables 1 and 2.

References throughout the specification to mutations in respect of BCL2, AOC1, BOC, DDX3X, CRYBG3, SLFN5, ETNK2, BRCA2, USP22, KIAA1683 or AP4E1 may, except for where the context requires otherwise, be taken as encompassing any of the mutations in respect of these various genes set out in Tables 1 and 2.

Similarly, references in the specification to mutations relevant in respect of a particular form of haematological malignancy may be interpreted as encompassing any of the mutations identified in Table 1 or Table 2 as being particularly associated with a cell line representative of the haematological malignancy in question.

TABLE 1

| Gene | Cell line | Cancer | Location | Consequence | Protein_position | Amino_acids | Codons |
|---|---|---|---|---|---|---|---|
| BCL2 | OCILY3 | DLBCL NOS (ABC) | chr18: 60985307 | missense_variant | 198 | A/V | gCa/gTa |
| BCL2 | OCILY19 | DLBCL NOS (GCB) | chr18: 60985302 | missense_variant | 200 | G/S | Ggt/Agt |
| BCL2 | SUDHL6 | DLBCL NOS (GCB) | chr18: 60985758 | missense_variant | 48 | I/F | Atc/Ttc |
| BCL2 | SUDHL6 | DLBCL NOS (GCB) | chr18: 60985515 | missense_variant | 129 | R/C | Cgc/Tgc |
| BCL2 | SUDHL4 | DLBCL NOS (GCB) | chr18: 60985725 | missense_variant | 59 | P/T | Cca/Aca |
| AOC1 | MM1S | Multiple myeloma | chr7: 150554261 | missense_variant | 235 | Q/E | Cag/Gag |
| AOC1 | OCILY19 | DLBCL NOS (GCB) | chr7: 150553895 | missense_variant | 113 | E/K | Gag/Aag |
| AOC1 | MM1R | multiple myeloma | chr7: 150554261 | missense_variant | 235 | Q/E | Cag/Gag |
| AOC1 | P3HR1 | burkitt lymphoma | chr7: 150554261 | missense_variant | 235 | Q/E | Cag/Gag |
| BOC | OCILY19 | DLBCL NOS (GCB) | chr3: 112991945 | missense_variant | 331 | Q/K | Cag/Aag |
| DDX3X | HBL1 | DLBCL NOS (ABC) | chrX: 41206603 | missense_variant | 603 | R/Q | cGa/cAa |
| DDX3X | SUDHL4 | DLBCL NOS (GCB) | chrX: 41205581 | missense_variant | 472 | H/L | cAt/cTt |
| DDX3X | SUDHL5 | DLBCL NOS (GCB) | chrX: 41205845 | missense_variant | 529 | I/L | Att/Ctt |
| DDX3X | SUDHL5 | DLBCL NOS (GCB) | chrX: 41205869 | missense_variant | 537 | N/D | Aac/Gac |
| DDX3X | OCILY7 | DLBCL NOS (GCB) | chrX: 41203490 | splice_acceptor_variant | — | — | — |
| CRYBG3 | SUDHL5 | DLBCL NOS (GCB) | chr3: 97596118 | missense_variant | 79 | H/R | cAt/cGt |
| CRYBG3 | SUDHL6 | DLBCL NOS (GCB) | chr3: 97634545 | missense_variant | 788 | A/P | Gca/Cca |
| ETNK2 | SUDHL5 | DLBCL NOS (GCB) | chr1: 204109237 | missense_variant | 265 | R/Q | cGg/cAg |
| SLFN5 | MM1S | Multiple myeloma | chr17: 33592491 | missense_variant | 754 | V/L | Gtc/Ctc |
| SLFN5 | MM1R | Multiple myeloma | chr17: 33592491 | missense_variant | 754 | V/L | Gtc/Ctc |
| SLFN5 | MM1R | Multiple myeloma | chr17: 33591546 | missense_variant | 495 | C/R | Tgt/Cgt |
| SLFN5 | MM1S | Multiple myeloma | chr17: 33591546 | missense_variant | 495 | C/R | Tgt/Cgt |
| SLFN5 | SUDHL5 | DLBCL NOS (GCB) | chr17: 33592320 | missense_variant | 697 | P/S | Ccc/Tcc |
| BRCA2 | MCCAR | burkitt lymphoma | chr13: 32972626 | stop_gained | 3326 | K/* | Aaa/Taa |
| BRCA2 | OCILY19 | DLBCL NOS (GCB) | chr13: 32906593 | missense_variant | 326 | S/R | agC/agA |
| FRG1B | EJM | Multiple myeloma | chr20: 29632660 | stop_gained | 159 | E/* | Gag/Tag |
| FRG1B | MM1S | Multiple myeloma | chr20: 29633900 | missense_variant | 180 | E/G | gAa/gGa |
| FRG1B | NAMALWA | burkitt lymphoma | chr20: 29633900 | missense_variant | 180 | E/G | gAa/gGa |
| FRG1B | WSUDLCL2 | DLBCL NOS (GCB) | chr20: 29633900 | missense_variant | 180 | E/G | gAa/gGa |
| FRG1B | SUDHL5 | DLBCL NOS (GCB) | chr20: 29632660 | stop_gained | 159 | E/* | Gag/Tag |
| FRG1B | SUDHL5 | DLBCL NOS (GCB) | chr20: 29633900 | missense_variant | 180 | E/G | gAa/gGa |
| FRG1B | AMO1 | Multiple myeloma | chr20: 29632660 | stop_gained | 159 | E/* | Gag/Tag |
| FRG1B | OCILY19 | DLBCL NOS (GCB) | chr20: 29633900 | missense_variant | 180 | E/G | gAa/gGa |
| FRG1B | GRANTA519 | Mantle cell | chr20: 29633900 | missense_variant | 180 | E/G | gAa/gGa |
| | | lymphoma | | | | | |
| FRG1B | SUDHL4 | DLBCL NOS (GCB) | chr20: 29633900 | missense_variant | 180 | E/G | gAa/gGa |
| KIAA1683 | SUDHL5 | DLBCL NOS (GCB) | chr19: 18367995 | missense_variant | 1367 | I/F | Atc/Ttc |
| KIAA1683 | OCILY19 | DLBCL NOS (GCB) | chr19: 18376863 | missense_variant | 496 | R/L | cGc/cTc |
| USP22 | OCILY19 | DLBCL NOS (GCB) | chr17: 20931872 | missense_variant | 96 | A/V | gCg/gTg |

TABLE 2

| | | | | Variant | Variant | | Protein |
|---|---|---|---|---|---|---|---|
| Gene | Cell line | Cancer | Chromosome | Classification | Type | Genome Change | Change |
| BCL2 | PFEIFFER | DLBCL NOS (GCB) | 18 | Silent | SNP | g.chr18: 60985876C > A | p.G8G |
| BCL2 | TOLEDO | DLBCL NOS (GCB) | 18 | Silent | SNP | g.chr18: 60985357C > G | p.L181L |
| BCL2 | KARPAS422 | DLBCL NOS (GCB) | 18 | Missense | SNP | g.chr18: 60985724G > A | p.P59L |
| | | | | Mutation | | | |
| BCL2 | DAUDI | Burkitt lymphoma | 18 | Silent | SNP | g.chr18: 60985521G > T | p.R127R |
| BCL2 | KARPAS422 | DLBCL NOS (GCB) | 18 | Silent | SNP | g.chr18: 60985351C > G | p.R183R |
| BCL2 | KARPAS422 | DLBCL NOS (GCB) | 18 | Missense | SNP | g.chr18: 60985760C > T | p.G47D |
| | | | | Mutation | | | |
| BCL2 | DOHH2 | DLBCL NOS (GCB) | 18 | De novo Start | SNP | g.chr18: 60986612A > C | |
| | | | | OutOfFrame | | | |
| BCL2 | DOHH2 | DLBCL NOS (GCB) | 18 | De novo Start | DEL | g.chr18: 60986384delA | |
| | | | | OutOfFrame | | | |
| BCL2 | DAUDI | Burkitt lymphoma | 18 | Silent | SNP | g.chr18: 60985549G > A | p.S117S |
| BCL2 | DAUDI | Burkitt lymphoma | 18 | Silent | SNP | g.chr18: 60985492C > T | p.E136E |
| BCL2 | DAUDI | Burkitt lymphoma | 18 | Silent | SNP | g.chr18: 60985399C > T | p.S167S |
| BCL2 | DAUDI | Burkitt lymphoma | 18 | Silent | SNP | g.chr18: 60985480G > A | p.D140D |
| BCL2 | DAUDI | Burkitt lymphoma | 18 | Silent | SNP | g.chr18: 60985410G > T | p.R164R |
| BCL2 | DAUDI | Burkitt lymphoma | 18 | Silent | SNP | g.chr18: 60985348G > A | p.H184H |
| BCL2 | WSUNHL | DLBCL NOS (GCB) | 18 | Missense | SNP | g.chr18: 60985890C > T | p.A4T |
| | | | | Mutation | | | |
| AOC1 | DOHH2 | DLBCL NOS (GCB) | 7 | Silent | SNP | g.chr7: 150554662G > A | p.Q368Q |
| BOC | RAJI | Burkitt lymphoma | 3 | Missense | SNP | g.chr3: 112991463G > C | p.E292Q |
| | | | | Mutation | | | |
| BOC | DOHH2 | DLBCL NOS (GCB) | 3 | Silent | SNP | g.chr3: 112993352G > A | p.V455V |
| DDX3X | SUDHL1 | Peripheral T cell | X | Missense | SNP | g.chrX: 41206195G > A | p.V567M |
| | | lymphoma (PTCL), | | Mutation | | | |
| | | anaplastic large | | | | | |
| | | cell lymphoma, | | | | | |
| | | ALK+ (ALK+ ALCL) | | | | | |
| DDX3X | DAUDI | Burkitt lymphoma | X | Silent | SNP | g.chrX: 41204526G > A | p.K373K |
| DDX3X | KARPAS422 | DLBCL NOS (GCB) | X | Missense | SNP | g.chrX: 41204447A > T | p.D347V |
| | | | | Mutation | | | |
| DDX3X | DAUDI | Burkitt lymphoma | X | Missense | SNP | g.chrX: 41206941A > G | p.N653S |
| | | | | Mutation | | | |
| DDX3X | DAUDI | Burkitt lymphoma | X | Silent | SNP | g.chrX: 41202597C > T | p.A224A |
| DDX3X | DOHH2 | DLBCL NOS (GCB) | X | Splice Site | SNP | g.chrX: 41203654T > C | |
| DDX3X | DAUDI | Burkitt lymphoma | X | Silent | SNP | g.chrX: 41204511T > C | p.D368D |
| DDX3X | DAUDI | Burkitt lymphoma | X | Silent | SNP | g.chrX: 41206933A > G | p.G650G |
| DDX3X | DAUDI | Burkitt lymphoma | X | Silent | SNP | g.chrX: 41202546A > G | p.Q207Q |
| DDX3X | DAUDI | Burkitt lymphoma | X | Silent | SNP | g.chrX: 41205606A > G | p.R480R |
| DDX3X | DAUDI | Burkitt lymphoma | X | Silent | SNP | g.chrX: 41205649T > C | p.L495L |
| DDX3X | ST486 | Burkitt lymphoma | X | Frame Shift Del | DEL | g.chrX: 41204541_41204542delTA | p.M380fs |
| DDX3X | RAJI | Burkitt lymphoma | X | Splice Site | SNP | g.chrX: 41203654T > G | |
| DDX3X | RAJI | Burkitt lymphoma | X | Silent | SNP | g.chrX: 41203518T > C | p.P297P |
| DDX3X | DAUDI | Burkitt lymphoma | X | Silent | SNP | g.chrX: 41202498C > T | p.I191I |
| CRYBG3 | DOHH2 | DLBCL NOS (GCB) | 3 | Missense | SNP | g.chr3: 97595897A > C | p.Q5H |
| | | | | Mutation | | | |
| ETNK2 | DOHH2 | DLBCL NOS (GCB) | 1 | Missense | SNP | g.chr1: 204110562A > C | p.V222G |
| | | | | Mutation | | | |
| SLFN5 | DOHH2 | DLBCL NOS (GCB) | 17 | Missense | SNP | g.chr17: 33591899C > A | p.N612K |
| | | | | Mutation | | | |
| SLFN5 | KARPAS299 | Peripheral T cell | 17 | Missense | SNP | g.chr17: 33591796G > A | p.G578D |
| | | lymphoma (PTCL), | | Mutation | | | |
| | | anaplastic large | | | | | |
| | | cell lymphoma, | | | | | |
| | | ALK+ (ALK+ ALCL) | | | | | |
| BRCA2 | NCIH929 | Multiple myeloma | 13 | Missense | SNP | g.chr13: 32937341A > G | p.R2668G |
| | | | | Mutation | | | |
| BRCA2 | DOHH2 | DLBCL NOS (GCB) | 13 | Missense | SNP | g.chr13: 32954261G > C | p.V3079L |
| | | | | Mutation | | | |
| BRCA2 | DAUDI | Burkitt lymphoma | 13 | Missense | SNP | g.chr13: 32906957C > T | p.R448C |
| | | | | Mutation | | | |
| FRG1B | RAJI | Burkitt lymphoma | 20 | Missense | DNP | g.chr20: 29625876_29625877CG > TA | p.A41T |
| | | | | Mutation | | | |
| USP22 | DAUDI | Burkitt lymphoma | 17 | Silent | SNP | g.chr17: 20946086C > A | p.P21P |
| USP22 | DOHH2 | DLBCL NOS (GCB) | 17 | Nonsense | SNP | g.chr17: 20910270T > A | p.K421* |
| | | | | Mutation | | | |
| USP22 | DAUDI | Burkitt lymphoma | 17 | Silent | SNP | g.chr17: 20946041C > T | p.Q36Q |
| USP22 | DAUDI | Burkitt lymphoma | 17 | Missense | SNP | g.chr17: 20946094C > G | p.A19P |
| | | | | Mutation | | | |
| USP22 | DAUDI | Burkitt lymphoma | 17 | Silent | SNP | g.chr17: 20907601A > G | p.F483F |
| AP4E1 | SUDHL1 | Peripheral T cell | 15 | Missense | SNP | g.chr15: 51201073C > T | p.S33F |
| | | lymphoma (PTCL), | | Mutation | | | |
| | | anaplastic large | | | | | |
| | | cell lymphoma, | | | | | |
| | | ALK+ (ALK+ ALCL) | | | | | |
| AP4E1 | DAUDI | Burkitt lymphoma | 15 | Silent | SNP | g.chr15: 51226869T > C | p.A306A |
| AP4E1 | DOHH2 | DLBCL NOS (GCB) | 15 | Silent | SNP | g.chr15: 51216153A > G | p.L124L |

Mutations in respect of BCL2 may be of particular relevance in respect of a haematological malignancy selected from the group consisting of: DLBCL NOS (GCB); DLBCL NOS (ABC); and Burkitt lymphoma.

A mutation in respect of BCL2 relevant in respect of the haematological malignancy DLBCL NOS (GCB) may be selected from the following:
a mutation that causes a change at position 200 of the gene product, such as a change from G to A at position 60985302 of chromosome 18, causing a change of G to S at position 200 of the gene product;
a mutation that causes a change at position 48 of the gene product, such as a change from A to T at position 60985758 of chromosome 18, causing a change of I to F at position 48 of the gene product;
a mutation that causes a change at position 129 of the gene product, such as a change from C to T at position 60985515 of chromosome 18, causing a change of R to C at position 129 of the gene product;
a mutation that causes a change at position 59 of the gene product, such as a change from C to A at position 60985725 of chromosome 18, causing a change of P to T at position 59 of the gene product;
a silent mutation at position 60985876 of chromosome 18, such as a change from C to A;
a silent mutation at position 60985357 of chromosome 18, such as a change from C to G;
a mutation that causes a change at position 59 of the gene product, such as a change from G to A at position 6098572 of chromosome 18, causing a change of P to L at position 59 of the gene product;
a silent mutation at position 60985351 of chromosome 18, such as a change from C to G;
a mutation that causes a change at position 47 of the gene product, such as a change from C to T at position 60985760 of chromosome 18, causing a change of G to D at position 47 of the gene product;
a mutation that causes the gene product not to be expressed, such as a change from A to C at position 60986612 of chromosome 18;
a mutation that causes the gene product not to be expressed, such as a deletion of A at position 60986384 of chromosome 18; and
a mutation that causes a change at position 4 of the gene product, such as a change from C to T at position 60985890 of chromosome 18, causing a change of A to T at position 4 of the gene product.

A mutation in respect of BCL2 relevant in respect of the haematological malignancy DLBCL NOS (ABC) may be selected from the following:
a mutation that causes a change at position 198 of the gene product, such as a change from C to T at position 60985307 of chromosome 18, causing a change of A to V at position 198 of the gene product.

A mutation in respect of BCL2 relevant in respect of the haematological malignancy Burkitt lymphoma may be selected from the following:
a silent mutation at position 60985521 of chromosome 18, such as a change from G to T;
a silent mutation at position 60985549 of chromosome 18, such as a change from G to A;
a silent mutation at position 60985492 of chromosome 18, such as a change from C to T;
a silent mutation at position 60985399 of chromosome 18, such as a change from C to T;
a silent mutation at position 60985480 of chromosome 18, such as a change from G to A;
a silent mutation at position 60985410 of chromosome 18, such as a change from G to T; and
a silent mutation at position 60985348 of chromosome 18, such as a change from G to A.

The inventors' finding that mutation of the BCL2 gene is associated with increased sensitivity to satraplatin is surprising, since such mutations have not previously been identified as markers for sensitivity to other platinum-based chemotherapeutic compounds, such as cisplatin. Indeed, many of the mutations identified by the inventors as indicating sensitivity of a haematological malignancy to treatment with satraplatin appear to be specific to sensitivity in respect of this agent, rather than platinum-based chemotherapeutic agents more generally. Mutations in respect of AOC1 may be of particular relevance in respect of a haematological malignancy selected from the group consisting of: multiple myeloma; DLBCL NOS (GCB); and Burkitt lymphoma.

A mutation in respect of AOC1 relevant in respect of the haematological malignancy multiple myeloma may be selected from the following:
a mutation that causes a change at position 235 of the gene product, such as a change from C to G at position 150554261 of chromosome 7, causing a change of Q to E at position 235 of the gene product (a mutation noted in two different multiple myeloma cell lines and in Burkitt lymphoma, as mentioned below).

A mutation in respect of AOC1 relevant in respect of the haematological malignancy DLBCL NOS (GCB) may be selected from the following:
a mutation that causes a change at position 113 of the gene product, such as a change from G to A at position 150553895 of chromosome 7, causing a change of E to K at position 113 of the gene product; and
a silent mutation at position 150554662 of chromosome 7, such as a change from G to A.

A mutation in respect of AOC1 relevant in respect of the haematological malignancy Burkitt lymphoma may be selected from the following:
a mutation that causes a change at position 235 of the gene product, such as a change from C to G at position 150554261 of chromosome 7, causing a change of Q to E at position 235 of the gene product (a mutation also noted in two different multiple myeloma cell lines).

Mutations in respect of BOC may be of particular relevance in respect of a haematological malignancy selected from the group consisting of: DLBCL NOS (GCB); and Burkitt lymphoma.

A mutation in respect of BOC relevant in respect of the haematological malignancy DLBCL NOS (GCB) may be selected from the following:
a mutation that causes a change at position 331 of the gene product, such as a change from C to A at position 112991945 of chromosome 3, causing a change of Q to K at position 331 of the gene product; and
a silent mutation at position 112993352 of chromosome 3, such as a change from G to A.

A mutation in respect of BOC relevant in respect of the haematological malignancy Burkitt lymphoma may be selected from the following:
a mutation that causes a change at position 292 of the gene product, such as a change from G to C at position 112991463 of chromosome 3, causing a change of E to Q at position 292 of the gene product.

Mutations in respect of DDX3X may be of particular relevance in respect of a haematological malignancy selected from the group consisting of: DLBCL NOS (ABC); DLBCL NOS (GCB); Burkitt lymphoma; and ALCL ALK+.

A mutation in respect of DDX3X relevant in respect of the haematological malignancy DLBCL NOS (ABC) may be selected from the following:
a mutation that causes a change at position 603 of the gene product, such as a change from G to A at position 41206603 of chromosome X, causing a change of R to Q at position 603 of the gene product.

A mutation in respect of DDX3X relevant in respect of the haematological malignancy DLBCL NOS (GCB) may be selected from the following:
a mutation that causes a change at position 472 of the gene product, such as a change from A to T at position 41205581 of chromosome X, causing a change of H to L at position 472 of the gene product;
a mutation that causes a change at position 529 of the gene product, such as a change from A to C at position 41205845 of chromosome X, causing a change of I to L at position 529 of the gene product;
a mutation that causes a change at position 537 of the gene product, such as a change from A to G at position 41205869 of chromosome X, causing a change of N to D at position 537 of the gene product;
a mutation that causes the gene at product not to be expressed, such as a change at position 41203490 of chromosome X causing formation of a splice acceptor variant; and
a mutation that causes the gene product not to be expressed, such as a change from T to C at position 41203654T of chromosome X causing alteration of a splice site.

A mutation in respect of DDX3X relevant in respect of the haematological malignancy Burkitt lymphoma may be selected from the following:
a silent mutation at position 41204526 of chromosome X, such as a change from G to A;
a mutation that causes a change at position 653 of the gene product, such as a change from A to G at position 41206941 of chromosome X, causing a change of N to S at position 653 of the gene product;
a silent mutation at position 41202597 of chromosome X, such as a change from C to T;
a silent mutation at position 41204511 of chromosome X, such as a change from T to C;
a silent mutation at position 41206933 of chromosome X, such as a change from A to G;
a silent mutation at position 41202546 of chromosome X, such as a change from A to G;
a silent mutation at position 41205606 of chromosome X, such as a change from A to G;
a silent mutation at position 41205649 of chromosome X, such as a change from T to C;
a mutation that causes the gene product not to be expressed, such as a deletion from position 41204541 to position 41204542 of chromosome X;
a mutation that causes the gene product not to be expressed, such as a change from T to G at position 41203654 of chromosome X;
a silent mutation at position 41203518 of chromosome X, such as a change from T to C;
a silent mutation at position 41202498 of chromosome X, such as a change from C to T.

A mutation in respect of DDX3X relevant in respect of the haematological malignancy PTCL, ALCL ALK+ may be selected from the following:
a mutation that causes a change position 567 of the gene product, such as a change from G to A at position 41206195 of chromosome X, causing a change of V to M at position 567 of the gene product.

Mutations in respect of CRYBG3 may be of particular relevance in respect of the haematological malignancy DLBCL NOS (GCB).

A mutation in respect of CRYBG3 relevant in respect of the haematological malignancy DLBCL NOS (GCB) may be selected from the following:
a mutation that causes a change at position 79 of the gene product, such as a change from A to G at position 97596118 of chromosome 3, causing a change of H to R at position 79 of the gene product;
a mutation that causes a change at position 788 of the gene product, such as a change from G to C at position 97634545 of chromosome 3, causing a change of A to P at position 788 of the gene product; and
a mutation that causes a change at position 5 of the gene product, such as a change from A to C at position 97595897 of chromosome 3, causing a change of Q to H at position 5 of the gene product.

Mutations in respect of ETNK2 may be of particular relevance in respect of the haematological malignancy DLBCL NOS (GCB).

A mutation in respect of ETNK2 relevant in respect of the haematological malignancy DLBCL NOS (GCB) may be selected from the following:
a mutation that causes a change at position 265 of the gene product, such as a change from G to A at position 204109237 of chromosome 1, causing a change of R to Q at position 265 of the gene product; and
a mutation that causes a change at position 222 of the gene product, such as a change from A to C at position 204110562 of chromosome 1, causing a change of V to G at position 222 of the gene product.

Mutations in respect of SLFN5 may be of particular relevance in respect of a haematological malignancy selected from the group consisting of: multiple myeloma; DLBCL NOS (GCB); and ALCL ALK+.

A mutation in respect of SLFN5 relevant in respect of the haematological malignancy multiple myeloma may be selected from the following:
a mutation that causes a change at position 754 of the gene product, such as a change from G to C at position 33592491 of chromosome 17, causing a change of V to L at position 754 of the gene product (a mutation noted in two different multiple myeloma cell lines); and
a mutation that causes a change at position 495 of the gene product, such as a change from T to C at position 33591546 of chromosome 17, causing a change of C to R at position 495 of the gene product (another mutation noted in two different multiple myeloma cell lines).

A mutation in respect of SLFN5 relevant in respect of the haematological malignancy DLBCL NOS (GCB) may be selected from the following:
a mutation that causes a change at position 697 of the gene product, such as a change from C to T at position 33592320 of chromosome 17, causing a change of P to S at position 697 of the gene product.
a mutation that causes a change at position 612 of the gene product, such as a change from C to A at position 33591899 of chromosome 17, causing a change of N to K at position 612 of the gene product.

A mutation in respect of SLFN5 relevant in respect of the haematological malignancy ALCL ALK+ may be selected from the following:
a mutation that causes a change at position 578 of the gene product, such as a change from G to A at position 33591796 of chromosome 17, causing a change of G to D at position 578 of the gene product.

Mutations in respect of BRCA2 may be of particular relevance in respect of a haematological malignancy selected from the group consisting of: Burkitt lymphoma; DLBCL NOS (GCB); and multiple myeloma.

A mutation in respect of BRCA2 relevant in respect of the haematological malignancy Burkitt lymphoma may be selected from the following:
a mutation that causes a truncation of the gene product, such as a change from A to T at position 32972626 of chromosome 13, causing truncation of the gene product at position 3326; and
a mutation that causes a change at position 448 of the gene product, such as a change from C to T at position 32906957 of chromosome 13, causing a change of R to C at position 448 of the gene product.

A mutation in respect of BRCA2 relevant in respect of the haematological malignancy DLBCL NOS (GCB) may be selected from the following:
a mutation that causes a change at position 326 of the gene product, such as a change from C to A at position 32906593 of chromosome 13, causing a change of S to R at position 326 of the gene product; and
a mutation that causes a change at position 3079 of the gene product, such as a change from G to C at position 32954261 of chromosome 13, causing a change of V to L at position 3079 of the gene product.

A mutation in respect of BRCA2 relevant in respect of the haematological malignancy multiple myeloma may be selected from the following:
a mutation that causes a change at position 2668 of the gene product, such as a change from A to G at position 32937341 of chromosome 13, causing a change of R to G at position 2668 of the gene product.

Mutations in respect of FRG1B may be of particular relevance in respect of a haematological malignancy selected from the group consisting of: multiple myeloma; Burkitt lymphoma; DLBCL NOS (GCB); and mantle cell lymphoma.

A mutation in respect of FRG1B relevant in respect of the haematological malignancy multiple myeloma may be selected from the following:
a mutation that causes truncation of the gene product, such as a change from G to T at position 29632660 of chromosome 20, causing truncation of the gene product at position 159 (a mutation noted in respect of two different multiple myeloma cell lines and a DLBCL NOS (GCB) cell line); and
a mutation that causes a change at position 180 of the gene product, such as a change from A to G at position 29633900 of chromosome 20, causing a change of E to G at position 180 of the gene product (a mutation also noted in respect of Burkitt lymphoma, four different DLBCL NOS (GCB) cell lines, and mantle cell lymphoma).

A mutation in respect of FRG1B relevant in respect of the haematological malignancy Burkitt lymphoma may be selected from the following:
a mutation that causes a change at position 180 of the gene product, such as a change from A to G at position 29633900 of chromosome 20, causing a change of E to G at position 180 of the gene product (a mutation also noted in respect of multiple myeloma, four different DLBCL NOS (GCB) cell lines, and mantle cell lymphoma); and
a mutation that causes a change at position 41 of the gene product, such as a change from CG to TA at positions 29625876 and 29625877 of chromosome 20, causing a change of A to T at position 41 of the gene product.

A mutation in respect of FRG1B relevant in respect of the haematological malignancy DLBCL NOS (GCB) may be selected from the following:
a mutation that causes a change at position 180 of the gene product, such as a change from A to G at position 29633900 of chromosome 20, causing a change of E to G at position 180 of the gene product (a mutation noted in respect of four different DLBCL NOS (GCB) cell lines, as well as multiple myeloma, Burkitt lymphoma, and mantle cell lymphoma); and
a mutation that causes truncation of the gene product, such as a change from G to T at position 29632660 of chromosome 20, causing truncation of the gene product at position 159 (a mutation also noted in respect of two different multiple myeloma cell lines).

A mutation in respect of FRG1B relevant in respect of the haematological malignancy mantle cell lymphoma may be selected from the following:
a mutation that causes a change at position 180 of the gene product, such as a change from A to G at position 29633900 of chromosome 20, causing a change of E to G at position 180 of the gene product (a mutation also noted in respect of multiple myeloma, Burkitt lymphoma, and four different DLBCL NOS (GCB) cell lines).

Mutations in respect of KIAA1683 may be of particular relevance in respect of the haematological malignancy DLBCL NOS (GCB).

A mutation in respect of KIAA1683 relevant in respect of DLBCL NOS (GCB) may be selected from the following:
a mutation that causes a change at position 1367 of the gene product, such as a change from A to T at position 18367995 of chromosome 19, causing a change of I to F at position 1367 of the gene product; and
a mutation that causes a change at position 496 of the gene product, such as a change from G to T at position 18376863 of chromosome 19, causing a change of R to L at position 496 of the gene product.

Mutations in respect of USP22 may be of particular relevance in respect of a haematological malignancy selected from the group consisting of: DLBCL NOS (GCB); and Burkitt lymphoma.

A mutation in respect of USP22 relevant in respect of DLBCL NOS (GCB) may be selected from the following:
a mutation that causes a change at position 96 of the gene product, such as a change from C to T at position 20931872 of chromosome 17, causing a change of A to V at position 96 of the gene product;
a mutation that causes a truncation of the gene product, such as a change from T to A at position 20910270 of chromosome 17; causing truncation of the gene product at position 421.

A mutation in respect of USP22 relevant in respect of Burkitt lymphoma may be selected from the following:
a silent mutation at position 20946086 of chromosome 17, such as a change from C to A;
a silent mutation at position 20946041 of chromosome 17, such as a change from C to T;
a mutation that causes a change at position 19 of the gene product, such as a change from C to G at position 20946094 of chromosome 17, causing a change of A to P at position 19 of the gene product; and
a silent mutation at position 20907601 of chromosome 17, such as a change from A to G.

Mutations in respect of AP4E1 may be of particular relevance in respect of a haematological malignancy selected from the group consisting of: ALCL ALK+; and Burkitt lymphoma; and DLBCL NOS (GCB).

A mutation in respect of AP4E1 relevant in respect of ALCL ALK+ may be selected from the following:
a mutation that causes a change at position 33 of the gene product, such as a change from C to T at position 51201073 of chromosome 15, causing a change of S to F at position 33 of the gene product.

A mutation in respect of AP4E1 relevant in respect of Burkitt lymphoma may be selected from the following:
a silent mutation at position 51226869 of chromosome 15, such as a change from T to C.

A mutation in respect of AP4E1 relevant in respect of DLBCL NOS (GCB) may be selected from the following:
a silent mutation at position 51216153 of chromosome 15, such as a change from A to G.

### Elevated Expression

As set out in the Examples below, as well as in various aspects of the invention already described, elevated expression of at least one gene selected from the group consisting of: TULP3; PAN3; AC073343.11.1; PHF2; KLHDC1; BRCA2; and WRN is associated with increased sensitivity of a haematological malignancy to treatment with satraplatin.

Elevated expression of a gene of interest may be determined with reference to elevated transcription and/or elevated translation. Thus, elevated expression may be demonstrated by an increase in mRNA indicative of gene transcription and/or an increase in the protein indicative of gene translation. Suitable examples of techniques by which elevated expression may be analysed and identified are set out in more detail below. Except for where the context requires otherwise, any of these suitable techniques may be performed to analyse elevated expression in respect of any of the genes in which this property may be of interest identified above.

In a suitable embodiment of the first, second, third, seventh, tenth, eleventh or thirteenth aspects of the invention, elevated expression may be analysed in respect of one or more of the following genes: TULP3 and/or PAN3 and/or AC073343.11.1 and/or PHF2 and/or KLHDC1 and/or BRCA2 and/or WRN.

Suitably, elevated expression may be analysed in respect of TULP3. Suitably, elevated expression may be analysed in respect of PAN3. Suitably, elevated expression may be analysed in respect of AC073343.11.1. Suitably, elevated expression may be analysed in respect of PHF2. Suitably, elevated expression may be analysed in respect of KLHDC1.

Suitably, elevated expression may be analysed in respect of BRCA2. Suitably, elevated expression may be analysed in respect of WRN. In each of these cases elevated expression may be analysed in respect of only the gene identified, or in respect of the gene identified in combination with one or more other genes. In each of these cases elevated expression may be present in respect of only the gene identified, or in respect of the gene identified in combination with one or more other genes.

Elevation of a gene of interest may be assessed with reference to any suitable comparator. The skilled person will be readily able to identify suitable comparators, which may (for example) include housekeeping genes, or expression levels of the gene of interest in healthy comparator tissues.

Elevated expression of a gene of interest may be assessed to be present, in the case that analysis indicates expression to be at least 5% greater than in a suitable comparator, at least 10% greater, at least 25% greater, at least 50% greater, at least 75% greater, at least 100% greater than in a suitable comparator. Merely by way of example, elevated expression may be assessed to be present when analysis indicates expression of the gene of interest to be at least 2-fold greater, at least 3-fold greater, at least 4-fold greater, at least 5-fold greater, or at least 10-fold greater than in a suitable comparator.

### Deficiency of a Gene

The fifth aspect of the invention relates to satraplatin for medical use in a haematological malignancy associated with deficiency of a gene selected from the group consisting of: MTAP; CDKN2B; CDKN2A; and DMRTA1.

The eleventh aspect of the invention relates to a method in which a gene located in the 9p21 locus may be analysed to determine if a deficiency is present (this being of assistance in determining how a haematological malignancy may respond to satraplatin treatment).

Analysis of genes located at the 9p21 locus (and particular of MTAP, CDKN2B, CDKN2A, or DMRTA1) for the presence of a deficiency also constitutes a suitable embodiment of other aspects of the invention, including (but not limited to) the first, second, third, and tenth aspects.

For present purposes, references to “deficiency” of a gene cover any means by which the function of the gene product is lost within cells. This may be by loss of the gene itself (for example by loss of copy number), or by changes within the gene or gene product that reduce function of the gene product.

In a suitable embodiment, a deficiency of a gene is selected from the group consisting of: a loss of copy numbers of the gene; a mutation or modification reducing transcription or translation of the gene; and a mutation reducing function of the gene product. Merely by way of example, a “stop” mutation (in which a novel stop codon is introduced by means of a mutation) represents a suitable embodiment of such a mutation.

Thus, in the case of an MTAP deficiency, this may be selected from the group consisting of: a loss of copy numbers of the MTAP gene; a mutation or modification reducing transcription or translation of the MTAP gene; and a mutation reducing function of the MTAP gene product.

In the case of a CDKN2B deficiency, this may be selected from the group consisting of: a loss of copy numbers of the CDKN2B gene; a mutation or modification reducing transcription or translation of the CDKN2B gene; and a mutation reducing function of the CDKN2B gene product.

In the case of a CDKN2A deficiency, this may be selected from the group consisting of: a loss of copy numbers of the CDKN2A gene; a mutation or modification reducing transcription or translation of the CDKN2A gene; and a mutation reducing function of the CDKN2A gene product.

In the case of a DMRTA1 deficiency, this may be selected from the group consisting of: a loss of copy numbers of the DMRTA1 gene; a mutation or modification reducing transcription or translation of the DMRTA1 gene; and a mutation reducing function of the DMRTA1 gene product.

Suitably, analysis for a deficiency may be conducted to identify a loss of copy numbers; and/or a mutation or modification reducing transcription or translation; and/or a mutation reducing function of the gene, such as MTAP. A deficiency may be determined to be present when one or more such changes is found.

Merely by way of example, a deficiency assessed with respect to a reduction in transcription or translation of gene of interest may cause a reduction of the relevant parameter by at least 5%, by at least 10%, by at least 20%, or by at least 30%. A deficiency may cause a reduction of the relevant parameter by at least 40%, at least 50%, at least 60%, at least 70% or more. In a suitable embodiment, a deficiency may be a total loss (100% reduction) as compared to a suitable comparator.

### Loss of Copy Number

From the above, it will be appreciated that a loss of copy number, or reduced copy number, in respect of a gene of interest is a highly relevant embodiment of a deficiency in respect of such a gene.

As shown in the Examples below, the inventors have found that a loss of copy number, or reduced copy number, in respect of a gene (or genes) of interest, may be associated with an increased sensitivity of a haematological malignancy to satraplatin. Suitably the loss of copy number may be a loss of copy number of at least one gene located in the 9p21 locus. Examples of such genes include those selected from the group consisting of: MTAP; CDKN2B; CDKN2A; and DMRTA1. Thus, a loss of copy number may be analysed in respect of MTAP and/or CDKN2B and/or CDKN2A and/or DMRTA1.

In a suitable embodiment, a loss of copy number is in respect of MTAP. In a suitable embodiment, a loss of copy number is in respect of CDKN2B. In a suitable embodiment, a loss of copy number is in respect of CDKN2A. In a suitable embodiment, a loss of copy number is in respect of DMRTA1. In each of these cases a loss of copy number may be analysed in respect of only the gene identified, or in respect of the gene identified in combination with one or more other genes. In each of these cases a loss of copy number may be present in respect of only the gene identified, or in respect of the gene identified in combination with one or more other genes.

The inventors have found that, in satraplatin-sensitive haematological malignancies, the prevalence of copy loss among the genes listed above is, from highest to lowest, is: MTAP followed by CDKN2B followed by CDKN2A followed by DMRTA1. Thus, in a suitable embodiment loss of copy number, or analysis of loss of copy number, may only be in respect of MTAP. In another suitable embodiment, loss of copy number, or analysis of loss of copy number, may be in respect of MTAP and CDKN2B. In a further suitable embodiment, loss of copy number, or analysis of loss of copy number, may be in respect of MTAP and CDKN2A. In a still further suitable embodiment, loss of copy number, or analysis of loss of copy number, may be in respect of MTAP and CDKN2A and CDKN2B.

Loss of copy number, or reduced copy number, in respect of one or more genes selected from the group consisting of: MTAP; CDKN2B; CDKN2A; and DMRTA1 may be associated with increased sensitivity to satraplatin in respect of haematological malignancies selected from the group consisting of: DLBCL; Burkitt lymphoma; multiple myeloma; CTCL; ALCL ALK+; and mantle cell lymphoma.

In the case of DLBCL or multiple myeloma, increased sensitivity to satraplatin may particularly be indicated by a loss of copy number, or reduced copy number, in respect of one or more genes selected from the group consisting of: MTAP; CDKN2B; and CDKN2A. Increased sensitivity of DLBCL or multiple myeloma to satraplatin may particularly be indicated by a loss of copy number, or reduced copy number, in respect of each of MTAP, CDKN2B and CDKN2A.

In the case of Burkitt lymphoma, CTCL, mantle cell lymphoma, or ALCL ALK+, increased sensitivity to satraplatin may particularly be indicated by a loss of copy number, or reduced copy number, in respect of each of MTAP, CDKN2B, CDKN2A and DMRTA1.

### Analysis of Genes

The invention makes use of the analysis of genes associated with cells of haematological malignancies in a number of different contexts. As discussed above, these include analysis for loss of the 9p21 locus, analysis for a deficiency (such as loss of gene copy number), analysis for the presence of mutations (either generally or specifically) within genes of interest, and analysis for elevated expression of a gene.

Analysis will be performed in respect of a suitable sample from a patient. Such a sample may, for example, be a cell of the haematological malignancy of interest. A suitable cell may, for example, be collected by means of a blood sample.

Results obtained by the chosen method of analysis may be compared with suitable reference values, to identify any gene-associated changes that are present.

### Analysis for Loss of the 9p21 Locus or for Loss of Copy Number

Analysis for loss of the 9p21 locus, or for loss of copy number in respect of a gene (or genes) of interest may be performed using any suitable techniques known to the skilled person. Examples of genes that may be investigated with respect to changes in their copy number are set out in elsewhere in the present specification.

Merely by way of example, suitable analysis may be performed using any of the following techniques: whole exome sequencing, whole genome sequencing, comparative genomic hybridization (including array comparative genomic hybridization), or fluorescent in situ hybridization.

### Analysis for Mutations of Genes of Interest

Analysis for mutations of a selected gene or genes, or to identify the presence of one or more specific mutations of interest, may be performed by any suitable means known to the skilled person. Examples of such genes, and of specific mutations that may be investigated in connection with the various aspects or embodiments of the invention, are set out in elsewhere in the present specification.

By way of illustration, suitable analysis may be performed using any of the following techniques: whole exome sequencing, whole genome sequencing.

### Analysis for Elevated Expression of Genes of Interest

Analysis for elevated expression of a gene of interest may be performed by any suitable means known to the skilled person. Examples of genes that may be analysed with reference to their elevated expression are set out in elsewhere in the present specification.

Elevated expression may be determined by analysing for the presence of increased mRNA indicative of increased gene transcription, or may be determined by analysing for the presence of increased levels of proteins indicative of increased translation to produce the gene product. In the case that analysis is to be conducted in respect of mRNA levels, analysis may be limited to only transcripts of the gene (or genes) of interest, or the entire transcriptome may be analysed, and results compared in respect of the gene (or genes) of interest.

Merely by way of example, suitable analysis may be performed using any of the following techniques: RNA sequencing, for example by next generation sequencing analysis; quantitative RT-PCR; and immunolabelling (carried out in respect of the relevant gene products).

### Analysis for a Deficiency in Respect of a Gene of Interest

The skilled person will be aware of many suitable techniques by which a deficiency in respect of a gene of interest may be identified. The skilled person may make use of any such suitable technique in order to practice the invention.

As referred to above, a deficiency in a gene of interest (including a gene located at the 9p21 locus) may arise for one or more reasons, including loss of copy numbers of the gene; a mutation or modification reducing transcription or translation of the gene; or a mutation reducing function of the gene product. Suitable techniques may be selected with reference to the nature of the deficiency. Merely by way of example, suitable analysis may be performed using any of the following techniques: RNA sequencing (as considered above), quantitative RT-PCR; immunolabelling.

### Cancers and Haematological Malignancies

The various aspects of the invention relate to methods of treatment, medical uses, patient stratification methods, diagnostic methods and biomarker panels that may be of use in the treatment of a range of cancers, in particular haematological malignancies. Unless the context requires otherwise, the considerations set out here in respect of cancers and haematological malignancies should be considered applicable to all aspects or embodiments of the present invention.

Suitably, references to “cancers” in the context of the present invention should, in addition to the haematological malignancies discussed below, also be taken to include non-haematological cancers selected from the group consisting of: glioblastoma; diffuse glioma; mesothelioma: melanoma; as well as oesophageal squamous cell cancer; bladder urothelial cancer; pancreatic adeno cancer; non-small cell lung cancer, head and neck squamous cell cancer; cholangiocarcinoma; GEJ adenocarcinoma; sarcoma; adrenal cortical cancer; renal cancer; invasive breast cancer; prostate cancer; and colorectal cancer.

However, the invention is particularly relevant with respect to haematological malignancies. Haematological malignancies encompass all blood cancers, and may include lymphomas, myelomas and leukaemias. In particular, the various aspects and embodiments of the invention may be of use in connection with lymphomas or myelomas.

In a suitable embodiment, the myeloma is a multiple myeloma.

In a suitable embodiment, a lymphoma is a mature B-cell lymphoma or a mature T-cell lymphoma.

In a suitable embodiment, a lymphoma or myeloma is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; and multiple myeloma.

A mature B-cell lymphoma may be selected from the group consisting of: plasma cell myeloma (multiple myeloma); mantle cell lymphoma; Burkitt lymphoma; diffuse large B cell lymphoma (DLBCL); DLBL NOS; DLBCL NOS germinal center B cell type (GCB); DLBCL NOS activated B cell type (ABC); primary DLBCL of the central nervous system (CNS); and secondary CNS lymphoma (SCNSL).

Suitably, a mature B-cell lymphoma may be a primary DLBCL of the central nervous system (CNS).

Suitably, a mature B-cell lymphoma may be a C5 type or MCD subtype DLBCL.

Suitably, said mature T-cell lymphoma may be selected from the group consisting of: peripheral T cell lymphoma (PTCL); PTCL NOS; anaplastic large T-cell lymphoma (ALCL); anaplastic large T-cell lymphoma; ALK⁺ (ALK⁺ ALCL); cutaneous T cell lymphoma (CTCL); Sézary syndrome; and a primary cutaneous CD30+ T cell lymphoproliferative disorder.

Suitably, said mature T-cell neoplasm may be a cutaneous T cell lymphoma (CTCL).

In the case of the haematological malignancy DLBCL NOS (GCB), increased sensitivity to satraplatin may be associated with the presence of one or more mutations in a gene selected from the group consisting of: BCL2; AOC1; BOC; DDX3X; CRYBG3; ETNK2; SLFN5; BRCA2; FRG1B; KIAA1683; USP22; and AP4E1 and/or increased sensitivity to satraplatin may be associated with a loss of copy number, or reduced copy number, in respect of one or more genes selected from the group consisting of: MTAP; CDKN2B; and CDKN2A.

In the case of the haematological malignancy DLBCL NOS (ABC), increased sensitivity to satraplatin may be associated with the presence of one or more mutations in a gene selected from the group consisting of: BCL2; and DDX3X.

In the case of the haematological malignancy Burkitt lymphoma, increased sensitivity to satraplatin may be associated with the presence of one or more mutations in a gene selected from the group consisting of: BCL2; AOC1; BOC; DDX3X; BRCA2; FRG1B; USP22; and AP4E1 and/or increased sensitivity to satraplatin may be associated with a loss of copy number, or reduced copy number, in respect of each of MTAP, CDKN2B, CDKN2A and DMRTA1.

In the case of the haematological malignancy ALCL ALK+, increased sensitivity to satraplatin may be associated with the presence of one or more mutations in a gene selected from the group consisting of: DDX3X; SLFN5; and AP4E1 and/or increased sensitivity to satraplatin may be associated with a loss of copy number, or reduced copy number, in respect of each of MTAP, CDKN2B, CDKN2A and DMRTA1.

In the case of the haematological malignancy multiple myeloma, increased sensitivity to satraplatin may be associated with the presence of one or more mutations in a gene selected from the group consisting of: BRCA2; and FRG1B and/or increased sensitivity to satraplatin may be associated with a loss of copy number, or reduced copy number, in respect of one or more genes selected from the group consisting of: MTAP; CDKN2B; and CDKN2A.

In the case of the haematological malignancy mantle cell lymphoma, increased sensitivity to satraplatin may be associated with the presence of one or more mutations in FRG1B and/or increased sensitivity to satraplatin may be associated with a loss of copy number, or reduced copy number, in respect of each of MTAP, CDKN2B, CDKN2A and DMRTA1.

In the case of the haematological malignancy CTCL, increased sensitivity to satraplatin may be associated with a loss of copy number, or reduced copy number, in respect of each of MTAP, CDKN2B, CDKN2A and DMRTA1.

### Satraplatin

Satraplatin (INN), is also known as JM-216, or as bis (acetato) ammine dichloro (cyclohexylamine) platinum (IV).

The molecular formula for satraplatin is C₁₀H₂₂N₂C₁₂O₄Pt, and its molecular weight is 500.29 Da. Satraplatin can be synthesized according to the method disclosed in U.S. Pat. Nos. 5,072,011 and 5,244,919 or by appropriate modification of the method disclosed in U.S. Pat. No. 6,518,428.

The chemical structure of satraplatin is:

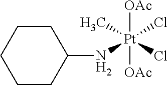

Satraplatin is a member of a class of platinum (IV) compounds that are absorbed by the oral route.

The lipophilic properties of these compounds, and hence their absorption, is believed to be largely determined by the nature of the axial acetate ligands. Unlike the square planar platinum (II) complexes cisplatin and carboplatin, satraplatin is an octahedral platinum (IV) compound.

### Forms of Satraplatin Suitable for Use in the Present Invention

Satraplatin for use in accordance with the various aspects and embodiments of the invention described herein may suitably be formulated for oral administration. By the same token, satraplatin when administered in methods of the invention may be administered orally.

### Doses and Therapeutically Effective Amounts of Satraplatin Suitable for Use in the Present Invention

The skilled person will be able to determine suitable dosages and dosage schedules based on publicly available information. It is understood that appropriate doses and dosage schedules may be determined by those or ordinary skill in the art, such as a physician. Accordingly, the following suggestions should be taken as being for guidance only.

Exemplary doses may be defined in the form of the milligram or microgram amount of satraplatin per kilogram body weight of a patient. Suitably, a dose of satraplatin defined in this matter may range from about 1 microgram per kilogram to about 500 milligrams per kilogram. For example, a suitable dose may be of between about 100 micrograms per kilogram to about 50 milligrams per kilogram. Merely by way of example, in the case of a patient of approximately 70 kg body weight, a suitable unit dose of satraplatin may be from about 0.05 mg to 2000 mg, such as from about 0.1 mg to 1000 mg.

A suitable unit dose may be administered 1, 2, 3 or more times per day. The unit dose may also be administered 1 to 7 times per week.

A person skilled in the art will further appreciate that doses can also be defined in the form of an amount of satraplatin to be administered per given area of body surface. In such definitions, a patient of 70 kg may be taken as having an approximate body surface area of 1.8 square meter. Suitable doses defined in this manner may be between about 50 microgram per square meter to about 15 grams per square meter, for example, about 5 milligrams per square meter to about 1.5 grams per square meter, or about 50 milligram per square meter to about 150 milligrams per square meter. The precise dose will ultimately be at the discretion of the attendant physician.

In a suitable embodiment, satraplatin may be for administration at a dosage schedule of 60-140 mg/m² po day 1-5 q 35 days. By way of example, the satraplatin may be for administration at a dosage schedule of 80 mg/m² po day 1-5 q 35 days. Alternatively, said satraplatin may be for administration at a dosage schedule of 60-80 mg/m² po day 1-5 q28 days.

Typical and preferred dosage schedules are 80 mg/m² po day 1-5 q 35 days, 100 mg/m² po day 1-5 q 35 days, 120 mg/m² po day 1-5 q 35 days or 140 mg/m² po day 1-5 q 35 days or 120, while lower doses such as 20 mg/m² or 40 mg/m² daily in combination therapies are further typical and preferred. In a further preferred embodiment, said satraplatin is administered at a dosage schedule of 80 mg/m² po day 1-5 q 35 days.

In an alternative embodiment, a typical and preferred dosage schedule may be 60-80 mg/m² po day 1-5 q 28 days. For example, the dosage schedule of 60 mg/m² po day 1-5 q 28 days, or 70 mg/m² po day 1-5 q 28 days, or 80 mg/m² po day 1-5 q 28 days may be preferred.

It will be appreciated that it may be necessary to make routine variations to the dosage amounts suggested above, depending on factors such as the age and weight of the patient, and the severity of the haematological malignancy or cancer to be treated.

### Patients

In the context of the present invention, references to a “patient” should be construed as encompassing both an individual undergoing treatment for a cancer (such as a haematological malignancy) or an individual that requires treatment, and for whom a suitable treatment regimen is being selected. It will be appreciated that the first construction is particularly suitable in respect of the first to ninth aspects of the invention, and the second construction is particularly suitable in respect of the tenth aspect of the invention.

A patient for the purposes of the present invention may suitably be a human.

### Treatment

For the purposes of the present disclosure, the terms “treatment” and “treating” should be taken as encompassing both therapeutic treatment and prophylactic or preventative measures. Treatment may be undertaken in order to prevent, slow down, or reduce an undesired physiological change or disorder, such as the growth, development or spread of cancer or haematological malignancy. For purposes of the present invention, beneficial or desired results include, but are not limited to, alleviation of symptoms, diminishment of extent of disease, stabilized state of disease (which is to say, disease that is not worsening), delay or slowing of disease progression, amelioration or palliation of the disease state, and remission (either partial or total).

Treatment may bring about prolonged survival as compared to expected survival if not receiving treatment. Alternatively, or additionally, treatment may provide a patient with an improved standard of life as compared to that which would be expected if not receiving treatment.

Those in need of treatment include individuals already with the disorder as well as those prone to have the disorder or those in whom the disorder is to be prevented.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Example 1—Satraplatin Demonstrates High Cytotoxic Activity in Genetically Defined Lymphoid Malignancies

### Materials and Methods

Cell lines and reagents. The cell lines listed in Table 3 were studied. All cell lines were cultured in appropriate media supplemented with 10-15% fetal bovine serum (Cat #FND500, ExCell Bio, China) at a temperature of 37° C., 5% CO2 and 95% humidity. Cell viability was measured using the CTG Luminescent Cell Viability Assay (Promega, Madison, WI, USA) by standard procedures as described below. Satraplatin was purchased from MedChemExpress (Shanghai, PRC) and cisplatin from Qilu Pharm (Jinan, Shandong, PRC), respectively. 1 mM stock solutions of both drugs were established for storage at −20° C. as previously described (5).

TABLE 3
List of cell lines analysed.

| No. | Cell Line Name | Tissue Origin |
|---|---|---|
| 1 | CA46 | Burkitt lymphoma |
| 2 | Daudi | Burkitt lymphoma |
| 3 | DOHH-2 | DLBCL NOS (GCB) |
| 4 | GRANTA-519 | Mantle Cell Lymphoma |
| 5 | HBL-1 | DLBCL NOS (ABC) |
| 6 | HuT 78 | Cutaneous T-cell Lymphoma |
| | | (CTCL), Sézary Lymphoma |
| 7 | JeKo-1 | Mantle Cell Lymphoma |
| 8 | KARPAS-299 | Peripheral T cell lymphoma |
| | | (PTCL), anaplastic large cell |
| | | lymphoma, ALK+ (ALK⁺ ALCL) |
| 9 | KARPAS-422 | DLBCL NOS (GCB) |
| 10 | L-82 | Peripheral T cell lymphoma |
| | | (PTCL), anaplastic large cell |
| | | lymphoma (ALCL) |
| 11 | NAMALWA | Burkitt lymphoma, EBV-related |
| 12 | NAMALWA CSN/70 | Burkitt lymphoma, EBV-related |
| 13 | OCI-LY-19 | DLBCL NOS (GCB) |
| 14 | OCI-LY3 | DLBCL NOS (ABC) |
| 15 | OCI-LY7 | DLBCL NOS (GCB) |
| 16 | P3HR-1 | Burkitt lymphoma |
| 17 | Pfeiffer | DLBCL NOS (GCB) |
| 18 | Raji | Burkitt lymphoma |
| 19 | Ramos | Burkitt lymphoma |
| 20 | ST486 | Burkitt lymphoma |
| 21 | SU-DHL-1 | Peripheral T cell lymphoma |
| | | (PTCL), anaplastic large cell |
| | | lymphoma , ALK+ (ALK⁺ ALCL) |
| 22 | SU-DHL-4 | DLBCL NOS (GCB) |
| 23 | SU-DHL-5 | DLBCL NOS (GCB) |
| 24 | SU-DHL-6 | DLBCL NOS (GCB) |
| 25 | SU-DHL-8 | DLBCL NOS (GCB) |
| 26 | Toledo | DLBCL NOS (GCB) |
| 27 | U-2932 | DLBCL NOS (ABC) |
| 28 | WSU-DLCL2 | DLBCL NOS (GCB) |
| 29 | WSU-NHL | DLBCL NOS (GCB) |
| 30 | Z-138 | Mantle Cell Lymphoma |
| 31 | AMO-1 | Multiple myeloma |
| 32 | EJM | Multiple myeloma |
| 33 | KMS-11 | Multiple myeloma |
| 34 | LP-1 | Multiple myeloma |
| 35 | MC/CAR | Burkitt lymphoma |
| 36 | MM.1R | Multiple myeloma |
| 37 | MM.1S | Multiple myeloma |
| 38 | MOLP8 | Multiple myeloma |
| 39 | NCI-H929 | Multiple myeloma |
| 40 | OPM2 | Multiple myeloma |
| 41 | RPMI 8226 | Multiple myeloma |
| 42 | U266B1 | Multiple myeloma |
| 43 | SK-LMS-1 | Vulva carcinoma |
| 44 | DU 145 | Prostate cancer |
| 45 | LNCaP clone FGC | Prostate cancer |
| 46 | 22Rv1 | Prostate cancer |
| 47 | OVCAR-8 | Ovary carcinoma |
| 48 | PC-3 | Prostate cancer |
| 49 | IGR-OV1 | Ovary carcinoma |
| 50 | HCC1428 | Breast cancer |
| 51 | HCC1937 | Breast cancer |
| 52 | MCF7 | Breast cancer |
| 53 | OVCAR-3 | Ovary carcinoma |
| 54 | L-363 | Multiple myeloma |
| 55 | A3/KAW | DLBCL NOS (GCB) |
| 56 | A549 | Lung cancer |
| 57 | NCI-H358 | Lung cancer |
| 58 | NCI-H3122 | Lung cancer |
| 59 | PC-9 | Lung cancer |
| 60 | SK-LU-1 | Lung cancer |
| 61 | TT | H&N/Thyroid cancer |
| 62 | NCI-H23 | Lung cancer |
| 63 | NCI-H1975 | Lung cancer |
| 64 | NCI-H1666 | Lung cancer |
| 65 | NCI-H1650 | Lung cancer |
| 66 | HCC827 | Lung cancer |

Determination of the half maximal inhibition concentration (IC50). Cells were harvested during the logarithmic growth period and cell numbers counted using Count-star (Inno-Alliance Biotech, USA). Cell density was adjusted to 4.44×10⁴ cells/mL with the respective culture medium. Then, 90 μL cell suspensions were added in triplicate to a 96-well plate with the final cell density of 4×10³ cells/well. After 24 h, 10 μL serial solutions of either cisplatin or satraplatin were transferred to each well in triplicate to achieve final drug concentrations per well of 100 μM, 31.6 μM, 10 μM, 3.16 μM, 1.0 μM, 316 nM, 100 nM, 31.6 nM and 10 nM, respectively. The test plate was incubated for 72 h in the humidified incubator at 37° C. with 5% CO₂, and then evaluated using CTG cell viability assay (Promega). For that purpose, plates were equilibrated at room temperature for approximately 30 min. Then, 50 μL CTG reagent was added to each well. The content was mixed for 5 min on an orbital shaker to induce cell lysis and the plate incubated at room temperature for 20 min to stabilize luminescent signal. Luminescence was recorded using EnVision Multi Label Reader (Perkin Elmer, Richmond, CA, USA).

Data Analysis. The data were analyzed and visualized using GraphPad Prism 5.0 (GraphPad Software, San Diego, CA, USA) and Think-Cell 11 (Think-Cell Software, Berlin, Germany). In order to calculate absolute IC50, a dose-response curve was fitted using a non-linear regression model with a sigmoidal dose response. The formula for calculating the cell survival rate was: Cell survival (%) at Cx=(Cx−M)/(N−M)×100%, Cx being the luminescence signal of cells cultured with any concentration of the test compound, M being the luminescence signal of the blank control (with no cells) and N being the vehicle control with culture medium containing 0.25% (v/v) dimethyl sulfoxide. The absolute IC50 was calculated according to the dose-response curve generated by GraphPad Prism 5.0. Raw cell viability data were then used and standard four-parameter monotonic log-logistic dose-response curves fitted using R package dr4pl (9). To investigate drug efficacy in the 66 test cell lines, the inventors computed several metrics from dose-response curves and used the values for the area under the curve (AUC) as a representation of drug efficacy. Efficacy among cell lines from different tissue origin was compared by analysis of variance, with p-values>0.05 representing no significant difference in sensitivity to the drug across test indications.

Whole-transcriptome sequencing (RNAseq). Total RNA extraction for baseline cell line samples was proceeded according to Qiagen Cat #74106 protocol (Qiagen, Hilden, Germany). The integrity of the total RNA was determined by a 2100 Bioanalyser (Agilent, Santa Clara, CA, USA) and quantified using NanoDrop (Thermo Fisher Scientific, Waltham, MA, USA). Only high-quality RNA samples (OD_{260/280}=1.8-2.2, OD_{260/230}≥2.0, RNA Integrity Number≥7, total RNA>500 ng) was used to construct the sequencing library. PolyA mRNA was purified from total RNA using oligo-dT-attached magnetic beads and then fragmented by fragmentation buffer. Taking these short fragments as templates, first-stranded cDNA was synthesized using reverse transcriptase and random primers, followed by second_stranded cDNA synthesis by standard measures. The synthesized cDNA was subjected to end-repair, phosphorylation and ‘A’ base addition according to a library construction protocol (TruSeq RNA Sample Preparation v2 Guide). Then, sequencing adapters were added to both size of the cDNA fragments. After polymerase chain reaction amplification for cDNA fragments, the targets of 250-350 bp were cleaned. After library construction, Qubit 2.0 fluorometer dsDNA HS Assay (Thermo Fisher Scientific) was used to quantify the concentration of the resulting sequencing libraries, while the size distribution was analyzed using an Agilent BioAnalyzer 2100. After library validation, Illumina cBOT cluster generation system with HiSeq PE Cluster Kits (Illumina, San Diego, CA, USA) was used to generate clusters. Paired-end sequencing was performed using Illumina NovaSeq 6000 platform following Illumina-provided protocols for 2×150 paired-end sequencing.

Gene expression analysis. The quality of RNAseq fastq raw reads was checked by FastQC software (Babraham Bioinformatics, https://www.bioinformatics.babraham.ac.uk/projects/fastqc/). The adapter and low-quality sequences were trimmed by Trimmomatic software (10). The reads were mapped to reference genes (ENSEMBL GRCh37.66) by Bowtie (11) software, and the gene expression was calculated by MMSEQ (12) software. The final expression values are log 2-transformed fragments per kilobase of exon model per million mapped fragment values. For each cohort of tested cell lines, the inventors started with gene expression matrix on 49,442 annotated genes for involved cell lines with expression data available. Expression of 49,442 genes was correlated with AUC values across 66 cell lines to identify positively correlated genes and negatively correlated genes to drug sensitivity. Spearman correlation coefficient<−0.55 and p<0.05 were used as cutoffs for genes correlated with enhanced sensitivity whereas Spearman correlation coefficient>0.55 and p<0.05 were used as cutoffs for genes correlated with resistance.

To study the genomic similarity and dissimilarity of cell lines that behaved differently in drug response, Principal Component Analysis (PCA) was performed on the complete transcriptomes of involved cell lines with expression data. Spearman correlation between gene expression and AUC across samples were calculated for all genes. The inventors identified differentially expressed (DE) genes between more sensitive and less sensitive groups by limma package (13), and performed gene ontology (GO) enrichment, pathway enrichment analysis on the DE genes. Gene set variation analysis (GSVA) (14) was performed on 2,057 well-curated gene sets derived from of MSigDB database (https://www.gsea-msigdb.org/gsea/msigdb).

Whole-exome sequencing (WES). Genomic DNA extraction from baseline cell line samples for WES was performed on KingFisher Flex (Thermo Fisher Scientific) with MagMAX™ DNA Multi-Sample Ultra 2.0 Kit (ABI, Waltham, MA, USA). Genomic DNA was quantified by Nanodrop™ 2000 spectrophotometer (Thermo Fisher Scientific). Only high-quality DNA (concentration>100 ng/μl, OD_{260/280}≥1.8-2.2, OD_{260/230}≥2) was used for library construction. Library construction was performed following the protocol of SureSelect XT HS2 DNA Reagent Kit (Agilent). In brief, fragmentation was first performed by ME220 Focused-ultrasonicator (Covaris, Woburn, MA, USA) after DNA quality control, followed by end-repair, dA-tailing and ligation. The ligation product was then purified using AMPure XP beads (Beckman Coulter, Indianapolis, IN, USA), the purified adaptor-ligated library was amplified, followed by another step of purification using AMPure XP beads (Beckman Coulter, Indianapolis, IN, USA). Finally, the purified WES libraries were analyzed by 2×150 paired-end sequencing [NovaSeq 6000 S2 Reagent Kit (Illumina)] on NovaSeq 6000 (Illumina) according to the manufacturer's protocol.

Gene mutation analysis. The quality of WES fastq raw data was checked by FastQC software. The adapter and low-quality sequences were trimmed by Trimmomatic software (10). The reads were aligned to the hg19 genome by BWA software (15), the variants called by GATK software (16) and annotated by VEP software (17). The driver status of mutations was predicted following the method of the CancerGenomeInterpreter database (18). Wilcoxon signed-rank test and Benjamini & Hochberg adjusted p-values were calculated between mutation status and efficacy endpoint (AUC values). The calculation for the difference in AUC between cell lines with mutated and those with wild-type gene was as follows: AUC difference=mean AUC for cell lines with somatic point mutation−mean AUC for cell lines with no somatic point mutation. AUC difference>0 indicates that a gene mutation is enriched in resistant cell lines (with high AUC) whereas an AUC difference<0 indicates that a gene mutation is enriched in sensitive cell lines (with low AUC). Maximal Information Coefficient (MIC) of each gene was computed between AUC and gene mutation status, and ratio of the mutation recurrent rate within the current cohort vs. the rate in all cell lines was calculated for each gene.

Analysis of copy number variation. Aligned sequences from WES data were used to estimate gene copy numbers (CN) by CopyWriteR R package (19). Genes with copy number values>3 were defined as a copy number amplification while copy number values<1 were defined as a copy number deletion. Wilcoxon signed-rank test and Benjamini & Hochberg adjusted p-values were calculated between amplification/deletion status and efficacy endpoint (AUC values). MIC of each gene was computed between AUC and amplification/deletion status, and ratio of the amplification/deletion recurrent rate within the current cohort vs. the rate in all cell lines was calculated for each gene.

### Results

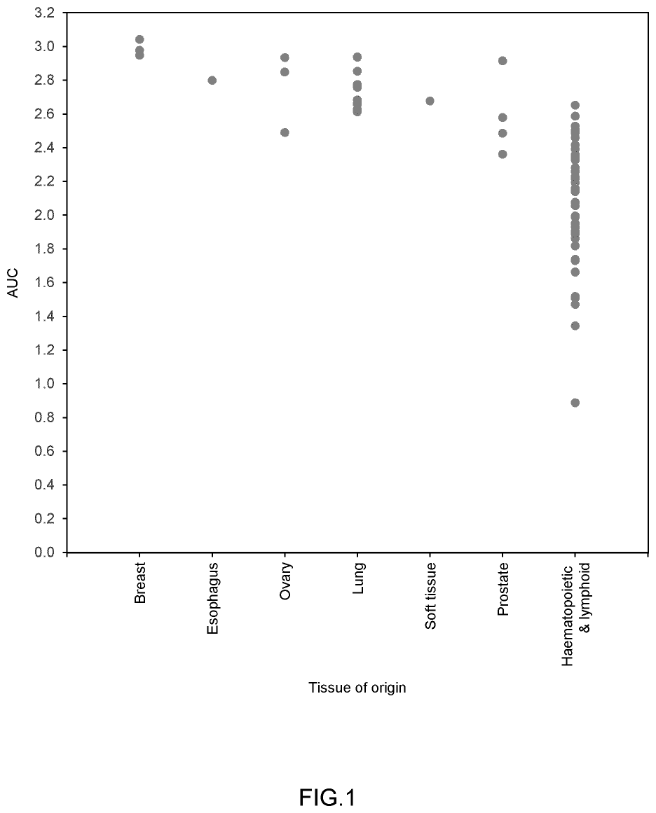

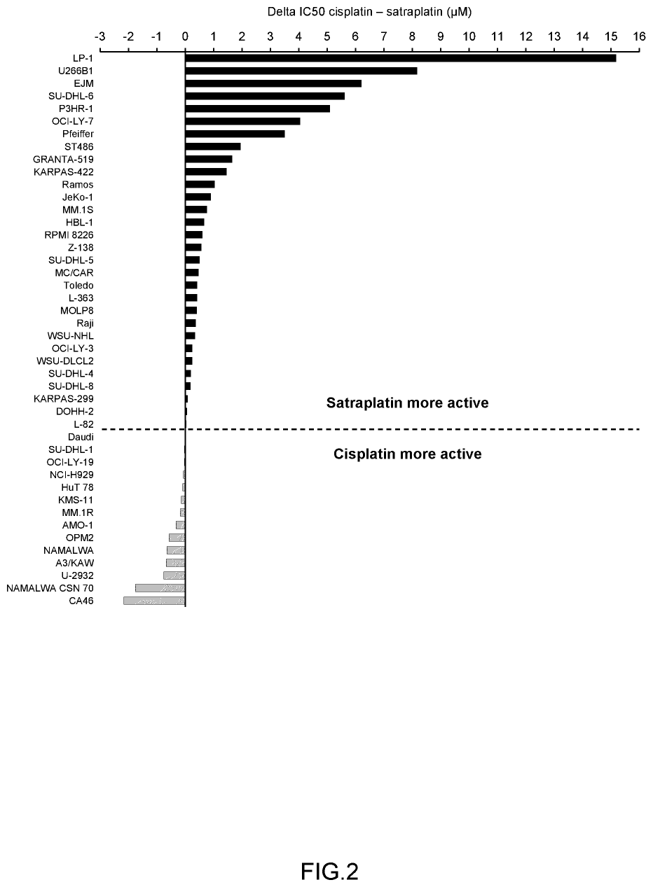

Satraplatin has high cytotoxic activity in lymphoid malignancies. The cytotoxic activity profile of satraplatin was tested in a standard cell-based assay of 66 cancer cell lines (Table 3) as described. To investigate and compare drug efficacy, AUC values as representation of drug efficacy were used. By analysis of variance (ANOVA), the inventors found significant differences in AUC values across all cancer types (FIG. 1) for satraplatin. Satraplatin was significantly more active in hematological malignancies compared to solid organ cancers (p-value=2.3e-08). Therefore, the cohort was split in solid organ cancers (n=22) and hematological malignancies (n=44) and only the latter one used for further analysis. Satraplatin was significantly more active within the group of hematologic malignancies than standard cisplatin (p-value=0.009) (FIG. 2) and achieved IC50 values at low to sub micromolar levels. When grouped by histology, satraplatin revealed high activity in diffuse large B-cell lymphoma (DLBCL), T-cell lymphoma, Mantle cell lymphoma (MCL), Multiple Myeloma (MM) and Burkitt lymphoma (BL) cell lines, respectively. In addition, statistical significances were calculated for some cell lines of special interest and it could be shown that especially for the cell line OCI-LY3 satraplatin is significantly more active than cisplatin with a p-value of 0.0006. Similar results were obtained for other DLBCL cell lines such as SU-DHHL-5 (p=0.003) and SU-DHL-6 (p=0.007).

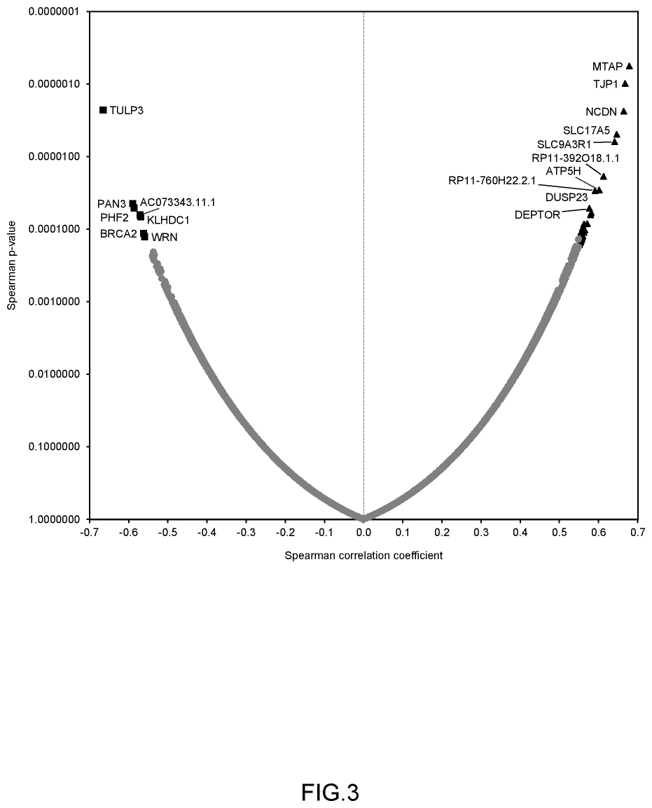

Identification of overexpressed genes leading to enhanced satraplatin sensitivity. Using gene expression analysis, seven genes were identified that positively correlated with enhanced satraplatin sensitivity with Spearman correlation coefficient<−0.55 and p-value<0.05 as cutoff (FIG. 3). The gene conferring the highest level of sensitivity was TULP3 followed by PAN3, PHF2, KLHDC1 and WRN, respectively. Other overexpressed genes known to be associated with platinum sensitivity such as BRCA2 were identified for both platinum compounds supporting the validity of the analysis. In total, 31 overexpressed genes leading to satraplatin resistance were identified, with MTAP achieving the highest Spearman correlation.

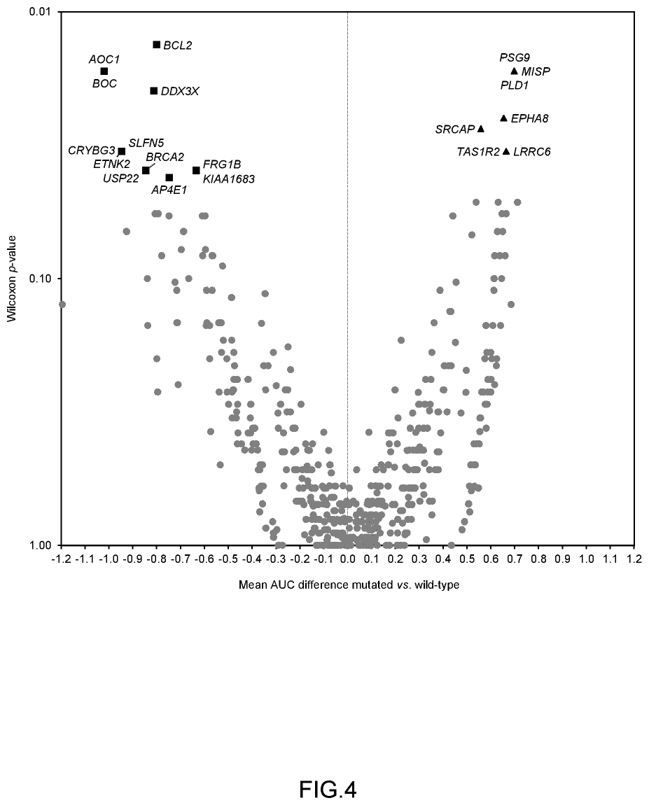

Single gene mutations leading to enhanced satraplatin sensitivity. On the single gene level, 19 genes were identified which either mediated an enhanced (12 genes) or reduced (7 genes) satraplatin cytotoxic activity when being mutated (FIG. 4). BCL2 apoptosis regulator turned out to be the single most significantly mutated gene linked to enhanced satraplatin cytotoxic activity. Of importance, the BCL2 gene mutation was not identified as marker for cisplatin activity and seems to be a unique hallmark for sensitivity to satraplatin. Additional gene mutations highlighting satraplatin sensitivity were AOC1, BOC, DDX3X, ETNK2 and USP22. Again, BRCA2 gene mutation was identified as sensitivity marker for both, satraplatin and cisplatin.

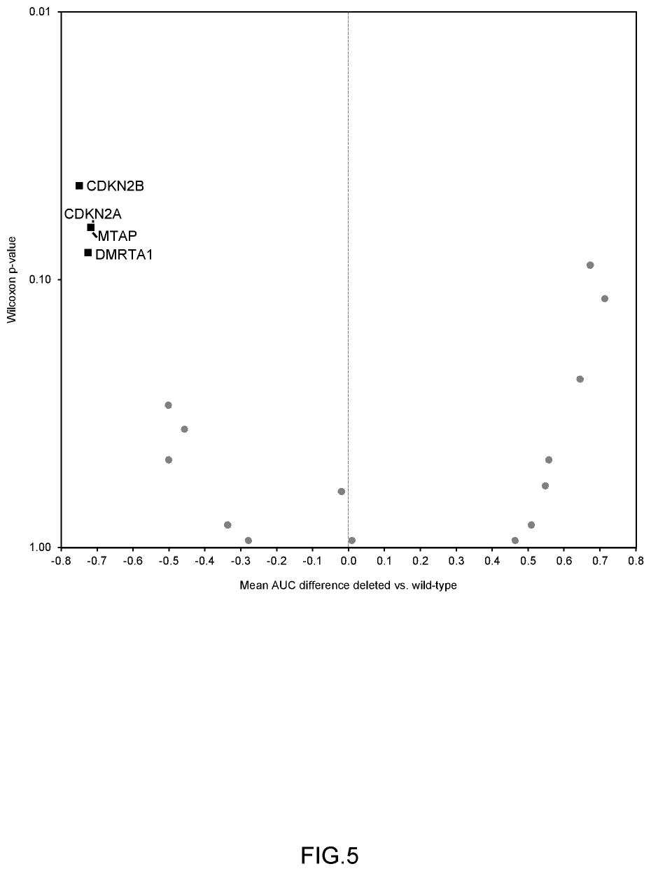

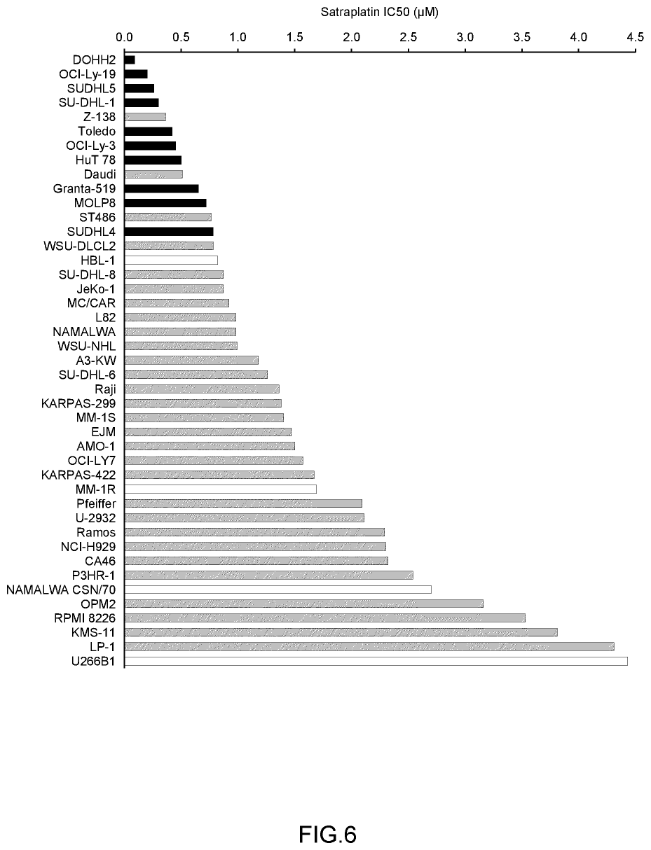

DNA copy number (CN) deletion analysis identifies recurrent chromosomal abnormalities of the 9p21 locus. Finally, copy number analysis was performed as described and 10 genes [chromosome 8 open reading frame 76 (C8orf76), collagen type XIV alpha 1 chain (COL14A 1), derlin 1 (DERL1), family with sequence similarity 83 member A (FAM83A), hyaluronan synthase 2 (HAS2), mitochondrial ribosomal protein L13 (MRPL13), zinc fingers and homeoboxes 2 (ZHX2), NBPF member 9 (NBPF9), phosphodiesterase 4D interacting protein (PDE4DIP), and peptidylprolyl isomerase A like 4G (PPIAL4G)] were identified by DNA CN amplification level that indicated a reduced satraplatin sensitivity. However, when DNA CN deletions were analysed, four genes (CDKN2A, CDKN2B, MTAP and DMRTA1) known to be located at the 9p21 locus could be identified that acted as biomarkers for enhanced satraplatin sensitivity (FIG. 5). Since the inventors had identified MTAP overexpression as marker of satraplatin resistance, they performed an in-depth analysis linking MTAP deficiency to satraplatin activity (FIG. 6). Plotting the IC50 values for satraplatin against MTAP status (defined using publicly available datasets), a significantly higher efficacy of satraplatin in MTAP deficient cell lines could be demonstrated (p=3.03e-07).

### Discussion

The inventors completed a systematic analysis on satraplatin activity in common hematological malignancies and identified DLBCL and its respective cell lines to be very sensitive to satraplatin with IC50 values at low micromolar or even sub-molar doses. Satraplatin is not only significantly superior in killing DLBCL cell lines such as OCI-LY7 cells when compared to cisplatin, but in addition and more importantly, satraplatin killed very efficiently the cell line OCI-LY3. The OCI-LY3 cell line is of special interest since it has a stable mutational pattern (20) including mutations of MYD88, PIM1, CD79B, CARD11 and PRDM1, respectively (Table 4). These mutations resemble the genetic landscape of the so-called Cluster 5 DLBCL as recently described by Chapuy and colleagues (21). Cluster 5 DLBCL genes include frequent BCL2 gains, concordant MYD88L265P/CD79B mutations, and additional mutations of ETV6, PIM1, GRHPR, TBL1XR1 and PRDM1. These mutations are typical for DLBCL patients with extranodal lymphoma involvement such as primary CNS lymphoma (PCNSL) and/or testicular lymphoma. As described in their pivotal publication, lymphoma tissue from eight of nine PCNSL patients fulfilled the C5 cluster criteria (Fisher's exact test, p<0.001) and laid the basis for the acceptance of said C5 genetic signature resembling PCNSL. Recently, the linkage between the C5 DLBCL cluster and PCNSL was confirmed by mutational profiling of DLBCL samples from additional 48 PCNSL patients at the MD Anderson Cancer Center. Therefore, and based on the mutational pattern, OCI-LY3 is suggested to be the most accepted PCNSL-like human DLBCL cell line, and highly suitable to predict and compare the efficacy of an investigational compound such as satraplatin for PCNSL treatment.

TABLE 4
Mutational pattern of OCI-LY3.

| | C5 | OCI-LY3 | PCNSL |
|---|---|---|---|
| | Signature | Cell | (profiling of |
| Mutation | (21) | Line | 48 patients)** |
| MYD88 | + | + | 81% |
| PIM1 | + | + | 55% |
| CD79B | + | + | 55% |
| CARD11 | − | + | 10% |
| ETV6 | + | ? | 10% |
| PRDM1 | + | + | 5% |

**Ou, A et al. Neuro-Oncology Advances (2020) 2: 1-8.

A similar DLBCL classification system based on genetic features was developed by Wright and colleagues (22). They have published a probabilistic classification of DLBCL patient samples and used the Chapuy data as a validation cohort. Again, this classification reveals genetic similarities between the different DLBCL subtypes. It is noteworthy that the MCD cohort as characterized by them shows major similarities with the C5 cohort as defined by Chapuy and colleagues regarding the mutational pattern. MCD DLBCL is primarily defined by MYD88L265P and CD79B mutations and known to spread secondarily to extranodal sites in 30% of all cases with CNS being the most common site. As for the C5 DLBCL cluster, MCD DLBCL is best represented by the OCI-LY3 cell line (22). In view of the data presented herein, satraplatin should be particularly useful in the treatment of DLBCL with primary extranodal lymphoma presentation, for example with lymphoma of the central nervous system, vitreo-retina, testis, and/or breast. The lipophilic characteristics of satraplatin enables the drug to access the cerebrospinal fluid at sufficient dose levels (23). In addition, its oral availability with a favorable side effect profile such as lack of neuro- and ototoxicity provide the rationale for further investigation of satraplatin in extranodal lymphomas with a special focus on PCNSL as outlined.

In a second step, the inventors set out for a more comprehensive molecular biomarker analysis to better understand the possible linkage between genetic characteristics of different lymphoma entities and satraplatin sensitivity. With the multitude of possible mechanisms of actions of satraplatin, a predictive biomarker is critical to better select appropriate patients who will benefit most. To their surprise, the inventors could clearly demonstrate meaningful differences between satraplatin and cisplatin as only few genetic alterations such as BRCA2 mutations or decreased BRCA2 expression were in common for both platinum compounds but most of the other genetic alterations identified clearly differentiated the two. When focusing on single gene mutation analysis, there is a particular satraplatin activity in cell lines that harbor either BCL2, USP22 or ETNK2 mutations, respectively. Interestingly, these mutations were not seen for cisplatin and especially the linkage to BCL2 mutations may be a potential explanation for the more severe hematotoxicity of satraplatin observed in previous clinical trials. Moreover, platinum resistance and refractoriness has been linked to the enhanced expression of anti-apoptotic genes such as BCL2 in solid organ tumor-derived cell lines (30). In that study, satraplatin in contrast to oxaliplatin was significantly more effective and able to reduce BCL2 expression up to 13-fold, independently of P53 mutational status. The BCL2-inhibiting function of satraplatin again establishes a link to hematological diseases in which BCL2 overexpression is a key driver, and BCL2 inhibition by venetoclax has shown tremendous clinical efficacy, e.g. for MCL, acute myeloid leukemia and chronic lymphocytic leukemia treatment (31).

Finally, satraplatin was more active on a wide variety of lymphoma cell lines harboring gene copy number deletions in the 9p21 locus including MTAP/CDKN2A/CDKN2B/DMRTA1. Cases with CDKN2A and/or CDKN2B deletions are frequent characteristics of different lymphoma entities such as T-cell lymphoma/T cell acute leukemia/Cutaneous T cell lymphoma (CTCL), DLBCL including PCNSL, MCL, and CLL. The methylthioadenosine phosphorylase (MTAP) gene is adjacent to the CDKN2A tumor suppressor gene and frequently co-deleted with CDKN2A, the most commonly deleted gene in human cancers including solid and hematologic malignancies. It occurs in approximately 15% of all cancers and deletion of MTAP has been reported to be linked to more aggressive tumors with shorter survival when compared to wild-type MTAP (24). MTAP deletion increases cellular concentrations of its substrate methylthioadenosin (MTA) and by competing with S-adenosyl-L-methionine (SAM), MTA binds to and partially inhibits Protein arginine methyltransferase 5 (PRMT5). Due to its essential role in the splicesosome machinery, normal PRMT5 function is particularly important for the hematopoietic compartment to maintain homologous recombination and DNA repair (25). As demonstrated in PRMT5 knockout mice, complete inhibition leads to severe, lethal pancytopenia (26). It has recently been shown that pharmacological inhibition of PRMT5 in MCL cell lines restarted a pro-apoptotic program and created a synergism with the bcl-2 inhibitor venetoclax (27). As described above, satraplatin has high activity in MTAP deleted hematopoietic cell lines, most likely because it causes severe DNA damaging lesions in the context of a homologous recombination-deficiency (functional PRMT inhibition). In addition, a key point could be the simultaneously observed unique BCL2 inhibition which specifically elicits superior activity compared to cisplatin.

Satraplatin is ideally suited to exploit the vulnerability in p16/CDKN2A and MTAP deficient hematologic malignancies and should be confirmable outside of lymphoid neoplasms in solid organ tumors. The inventors postulate that satraplatin is a preferred platinum for the treatment of tumors harboring the indicated gene mutations, reduced gene expression and, in particular, in 9p21 deleted/methylated tumors. Within the lymphoma entities, satraplatin is of particular interest for the treatment of PCNSL and CTCL. For PCNSL, de/9p21 and/or BCL2 mutations are present in nearly 80% of cases (28). For CTCL, deletion of CDKN2A/CDKN2B has been described as a hallmark in most subtypes like Mycosis Fungoides (MF) and Sezary Syndrome (SS). This deletion is a continuum that occurs more frequently with disease progression and is associated with more aggressive clinical behavior (29). There is an increasing loss of CDKN2A/B copy numbers and, thus, MTAP deletion in localized forms of MF when progressing towards systemic SS. Again, the high efficacy of satraplatin with low-micromolar IC50 in the representative CTCL cell line HUT78 is supporting this notion. One can assume that also in this disease—such as in PCNSL—the lipophilic characteristic of satraplatin is well suited and anticipates high treatment efficacy. Following these data, clinical trials in both entities are in preparation.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

1 Edward Chu; Vincent T. DeVita Jr. Goyena R and Fallis A.: Physicians' Cancer Chemotherapy Drug Manual 2019, 2019.
2 Pabla N and Dong Z: Cisplatin nephrotoxicity: mechanisms and renoprotective strategies. Kidney Int 73: 994-1007, 2008. PMID: 18272962. DOI: 10.1038/sj.ki.5002786.
3 Doshi G, Sonpavde G and Sternberg C N: Clinical and pharmacokinetic evaluation of satraplatin. Expert Opin Drug Metab Toxicol 8: 103-111, 2012. DOI: 10.1517/17425255.2012.636352.
4 Bhargava A and Vaishampayan U N: Satraplatin: Leading the new generation of oral platinum agents. Expert Opin Investig Drugs 18: 1787-1797, 2009. DOI: 10.1517/13543780903362437.
5 Wosikowski K, Lamphere L, Unteregger G, Jung V, Kaplan F, Xu J P, Rattel B and Caligiuri M: Preclinical antitumor activity of the oral platinum analog satraplatin. Cancer Chemother Pharmacol 60: 589-600, 2007. DOI: 10.1007/s00280-007-0502-z.
6 Sova P, Mistr A, Kroutil A, Zak F, Pouckova P and Zadinova M: Comparative anti-tumor efficacy of two orally administered platinum(IV) drugs in nude mice bearing human tumor xenografts. Anticancer Drugs 17: 201-206, 2006. PMID: 16428939. DOI: 10.1097/00001813-200602000-00012.
7 Latif T, Wood L, Connell C, Smith D C, Vaughn D, Lebwohl D and Peereboom D: Phase II study of oral bis (aceto) ammine dichloro (cyclohexamine) platinum (IV) (JM-216, BMS-182751) given daily ×5 in hormone refractory prostate cancer (HRPC). Invest New Drugs 23: 79-84, 2005. PMID: 15528984. DOI: 10.1023/B:DRUG.0000047109.76766.84.
8 Sternberg C N, Petrylak D P, Sartor O, Witjes J A, Demkow T, Ferrero J M, Eymard J C, Falcon S, Calabrò F, James N, Bodrogi I, Harper P, Wirth M, Berry W, Petrone M E, McKearn T J, Noursalehi M, George M and Rozencweig M: Multinational, double-blind, phase Ill study of prednisone and either satraplatin or placebo in patients with castrate-refractory prostate cancer progressing after prior chemotherapy: The SPARC trial. J Clin Oncol 27: 5431-5438, 2009. DOI: 10.1200/JCO.2008.20.1228.
9 Ritz C, Baty F, Streibig J C and Gerhard D: Dose-Response Analysis Using R. PLoS One 10: e0146021, 2015. PMID: 26717316. DOI: 10.1371/journal.pone.0146021.
10 Bolger A M, Lohse M and Usadel B: Trimmomatic: a flexible trimmer for Illumina sequence data. Bioinformatics 30: 2114-2120, 2014. PMID: 24695404. DOI: 10.1093/bioinformatics/btu170.
11 Langmead B, Trapnell C, Pop M and Salzberg S L: Ultrafast and memory-efficient alignment of short DNA sequences to the human genome. Genome Biol 10: R25, 2009.

PMID: 19261174. DOI: 10.1186/gb-2009-10-3-r25.
12 Turro E, Su S-Y, Gonçalves Â, Coin L J M, Richardson S and Lewin A: Haplotype and isoform specific expression estimation using multi-mapping RNA-seq reads. Genome Biol 12: R13, 2011. PMID: 21310039. DOI: 10.1186/gb-2011-12-2-r13.
13 Ritchie M E, Phipson B, Wu D, Hu Y, Law C W, Shi W and Smyth G K: limma powers differential expression analyses for RNA-sequencing and microarray studies. Nucleic Acids Res 43: e47, 2015. PMID: 25605792. DOI: 10.1093/nar/gkv007.
14 Hanzelmann S, Castelo R and Guinney J: GSVA: gene set variation analysis for microarray and RNA-seq data. BMC Bioinformatics 14: 7, 2013. PMID: 23323831. DOI: 10.1186/1471-2105-14-7.
15 Li H: Aligning sequence reads, clone sequences and assembly contigs with BWA-MEM. arXiv Prepr arXiv13033997, 2013.
16 McKenna A, Hanna M, Banks E, Sivachenko A, Cibulskis K, Kernytsky A, Garimella K, Altshuler D, Gabriel S, Daly M and DePristo M A: The Genome Analysis Toolkit: a MapReduce framework for analyzing next-generation DNA sequencing data. Genome Res 20: 1297-1303, 2010. PMID: 20644199. DOI: 10.1101/gr.107524.110.
17 McLaren W, Gil L, Hunt S E, Riat H S, Ritchie G R S, Thormann A, Flicek P and Cunningham F: The Ensembl Variant Effect Predictor. Genome Biol 17:122, 2016. DOI: 10.1186/s13059-016-0974-4.
18 Tamborero D, Rubio-Perez C, Deu-Pons J, Schroeder M P, Vivancos A, Rovira A, Tusquets I, Albanell J, Rodon J, Tabernero J, de Torres C, Dienstmann R, Gonzalez-Perez A and Lopez-Bigas N: Cancer Genome Interpreter annotates the biological and clinical relevance of tumor alterations. Genome Med 10: 25, 2018. PMID: 29592813. DOI: 10.1186/s13073-018-0531-8.
19 Kuilman T, Velds A, Kemper K, Ranzani M, Bombardelli L, Hoogstraat M, Nevedomskaya E, Xu G, de Ruiter J, Lolkema M P, Ylstra B, Jonkers J, Rottenberg S, Wessels L F, Adams D J, Peeper D S and Krijgsman O: CopywriteR: DNA copy number detection from off-target sequence data. Genome Biol 16: 49, 2015. PMID: 25887352. DOI: 10.1186/s13059-015-0617-1.
20 Juskevicius D, Müller A, Hashwah H, Lundberg P, Tzankov A and Menter T: Characterization of the mutational profile of 11 diffuse large B-cell lymphoma cell lines. Leuk Lymphoma 59: 1710-1716, 2018. DOI: 10.1080/10428194.2017.1387903.
21 Chapuy B, Stewart C, Dunford A J, Kim J, Kamburov A, Redd R A, Lawrence M S, Roemer M G M, Li A J, Ziepert M, Staiger A M, Wala J A, Ducar M D, Leshchiner I, Rheinbay E, Taylor-Weiner A, Coughlin C A, Hess J M, Pedamallu C S, Livitz D, Rosebrock D, Rosenberg M, Tracy A A, Horn H, Van Hummelen P, Feldman A L, Link B K, Novak A J, Cerhan J R, Habermann T M, Siebert R, Rosenwald A, Thorner A R, Meyerson M L, Golub T R, Beroukhim R, Wulf G G, Ott G, Rodig S J, Monti S, Neuberg D S, Loeffler M, Pfreundschuh M, Trümper L, Getz G and Shipp M A: Molecular subtypes of diffuse large B cell lymphoma are associated with distinct pathogenic mechanisms and outcomes. Nat Med 24: 679-690, 2018. DOI: 10.1038/s41591-018-0016-8.
22 Wright G W, Huang D W, Phelan J D, Coulibaly Z A, Roulland S, Young R M, Wang J Q, Schmitz R, Morin R D, Tang J, Jiang A, Bagaev A, Plotnikova O, Kotlov N, Johnson C A, Wilson W H, Scott D W and Staudt L M: A Probabilistic Classification Tool for Genetic Subtypes of Diffuse Large B Cell Lymphoma with Therapeutic Implications. Cancer Cell 37: 551-568.e14, 2020. PMID: 32289277. DOI: 10.1016/j.ccell.2020.03.015.
23 Marcus L, Murphy R, Fox E, McCully C, Cruz R, Warren K E, Meyer T, McNiff E, Balis F M and Widemann B C: The plasma and cerebrospinal fluid pharmacokinetics of the platinum analog satraplatin after intravenous administration in non-human primates. Cancer Chemother Pharmacol 69: 247-252, 2012. DOI: 10.1007/s00280-011-1659-z.
24 Bertino J R, Waud W R, Parker W B and Lubin M: Targeting tumors that lack methylthioadenosine phosphorylase (MTAP) activity: current strategies. Cancer Biol Ther 11: 627-632, 2011. PMID: 21301207. DOI: 10.4161/cbt.11.7.14948.
25 Hamard P-J, Santiago G E, Liu F, Karl D L, Martinez C, Man N, Mookhtiar A K, Duffort S, Greenblatt S, Verdun R E and Nimer S D: PRMT5 Regulates DNA Repair by Controlling the Alternative Splicing of Histone-Modifying Enzymes. Cell Rep 24: 2643-2657, 2018. PMID: 30184499. DOI: 10.1016/j.celrep.2018.08.002.
26 Liu F, Cheng G, Hamard P-J, Greenblatt S, Wang L, Man N, Perna F, Xu H, Tadi M, Luciani L and Nimer S D: Arginine methyltransferase PRMT5 is essential for sustaining normal adult hematopoiesis. J Clin Invest 125: 3532-3544, 2015. PMID: 26258414. DOI: 10.1172/JC181749.
27 Brown F, Hwang I, Sloan S, Hinterschied C, Helmig-mason J, Long M, Youssef Y, Chan W, Prouty A, Zhang Y, Vaddi K, Chen-kiang S, Diliberto M, Elemento O, Sehgal L, Alinari L, Scherle P, Lapalombella R, Paik J and Baiocchi R A: PRMT5 Inhibition Restarts a Pro-Apoptotic Program and Creates Vulnerability to Combination Treatment with BCL-2 Inhibitor Venetoclax in Mantle Cell Lymphoma, 2021.
28 Schwindt H, Vater I, Kreuz M, Montesinos-Rongen M, Brunn A, Richter J, Gesk S, Ammerpohl O, Wiestler O D, Hasenclever D, Deckert M and Siebert R: Chromosomal imbalances and partial uniparental disomies in primary central nervous system lymphoma. Leukemia 23: 1875-1884, 2009. PMID: 19494841. DOI: 10.1038/leu.2009.120.
29 Laharanne E, Chevret E, Idrissi Y, Gentil C, Longy M, Ferrer J, Dubus P, Jouary T, Vergier B, Beylot-Barry M and Merlio J-P: CDKN2A-CDKN2B deletion defines an aggressive subset of cutaneous T-cell lymphoma. Mod Pathol an Off J United States Can Acad Pathol Inc 23: 547-558, 2010. PMID: 20118908. DOI: 10.1038/modpathol.2009.196.
30 Kalimutho M, Minutolo A, Grelli S, Formosa A, Sancesario G, Valentini A, Federici G and Bernardini S: Satraplatin (JM-216) mediates G2/M cell cycle arrest and potentiates apoptosis via multiple death pathways in colorectal cancer cells thus overcoming platinum chemo-resistance. Cancer Chemother Pharmacol, 2011. DOI: 10.1007/s00280-010-1428-4.
31 Al-Sawaf O, Zhang C, Tandon M, Sinha A, Fink A-M, Robrecht S, Samoylova O, Liberati A M, Pinilla-lbarz J, Opat S, Sivcheva L, Le Dû K, Fogliatto L M, Niemann C U, Weinkove R, Robinson S, Kipps T J, Tausch E, Schary W, Ritgen M, Wendtner C-M, Kreuzer K-A, Eichhorst B, Stilgenbauer S, Hallek M and Fischer K: Venetoclax plus obinutuzumab versus chlorambucil plus obinutuzumab for previously untreated chronic lymphocytic leukaemia (CLL14): follow-up results from a multicentre, open-label, randomised, phase 3 trial. Lancet Oncol 21: 1188-1200, 2020. PMID: 32888452. DOI: 10.1016/S1470-2045(20)30443-5.

### Illustrative Embodiments of the Invention

The invention will further be illustrated with reference to the following numbered embodiments.
1. Satraplatin for use in the treatment of a haematological malignancy in a patient identified as likely to benefit from such treatment by analysis of a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN.
2. Satraplatin for use according to embodiment 1, wherein the haematological malignancy is a lymphoma or myeloma.
3. Satraplatin for use according to embodiment 2, wherein the lymphoma or myeloma is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; and multiple myeloma.
4. Satraplatin for use according to any of embodiments 1 to 3, wherein the gene is one located in the 9p21 locus, and the analysis identifies reduced copy numbers of the gene.
5. Satraplatin for use according to embodiment 4, wherein the gene is selected from the group consisting of: MTAP; CDKN2B; CDKN2A; and DMRTA1, and the analysis identifies a deficiency of the gene.
6. Satraplatin for use according to embodiment 5, wherein the deficiency of the gene is a reduced copy number of the gene.
7. Satraplatin for use according to any of embodiments 4 to 6, wherein the haematological malignancy is selected from the group consisting of: Burkitt lymphoma; DLBCL; CTCL; multiple myeloma; ALCL ALK+; and mantle cell lymphoma.
8. Satraplatin for use according to any of embodiments 1 to 3, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.
9. Satraplatin for use according to embodiment 8, wherein the mutation is selected from those set out in Table 1 and Table 2.
10. Satraplatin for use according to embodiment 8 or embodiment 9, wherein the haematological malignancy is selected from the group consisting of: Burkitt lymphoma; DLBCL; and ALCL ALK+.
11. Satraplatin for use according to any of embodiments 1 to 3, wherein the gene is selected from the group consisting of: TULP3; PAN3; AC073343.11.1; PHF2; KLHDC1; BRCA2; and WRN, and the analysis identifies elevated expression of the gene.
12. Satraplatin for use in the treatment of a haematological malignancy with a loss of the 9p21 locus.
13. Satraplatin for use in the treatment of a haematological malignancy with a deficiency of a gene selected from the group consisting of: MTAP; CDKN2B; CDKN2A; and DMRTA1.
14. Satraplatin for use according to embodiment 13, wherein the deficiency is reduced copy number of the gene selected from the group consisting of: MTAP; CDKN2B; CDKN2A; and DMRTA1.
15. Satraplatin for use according to any of embodiments 12 to 14, wherein the haematological malignancy is selected from the group consisting of: Burkitt lymphoma; DLBCL; CTCL; multiple myeloma; ALCL ALK+; and mantle cell lymphoma.
16. Satraplatin for use in the treatment of a haematological malignancy with mutation of a gene selected from the group consisting of: BCL2; AOC1; BOC; DDX3X; CRYBG3; SLFN5; ETNK2; BRCA2; USP22; KIAA1683; and AP4E1.
17. Satraplatin for use according to embodiment 16, wherein the mutation is selected from the group set out in Table 1 and Table 2.
18. Satraplatin for use according to embodiment 16 or embodiment 17, wherein the haematological malignancy is selected from the group consisting of: Burkitt lymphoma; DLBCL; and ALCL ALK+.
19. Satraplatin for use in the treatment of a haematological malignancy with elevated expression of TULP3; PAN3; AC073343.11.1; PHF2; KLHDC1; BRCA2; or WRN.
20. A method of determining the likelihood of a haematological malignancy responding favourably to satraplatin treatment, the method comprising:
analysing a cell of the haematological malignancy for a change in a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; wherein
identification of a change in the gene analysed indicates that the haematological malignancy is likely to respond favourably to satraplatin treatment.
21. A method according to embodiment 20, wherein the gene is one located in the 9p21 locus, and the analysis identifies a deficiency of the gene.
22. A method according to embodiment 21, wherein the gene is one located in the 9p21 locus, and the analysis identifies reduced copy numbers of the gene.
23. A method according to embodiment 21 or embodiment 22, wherein the gene is selected from the group consisting of: MTAP; CDKN2B; CDKN2A; and DMRTA1.
24. A method according to any of embodiments 20 to 23, wherein the haematological malignancy is selected from the group consisting of: Burkitt lymphoma; DLBCL; CTCL; multiple myeloma; ALCL ALK+; and mantle cell lymphoma.
25. A method according to embodiment 20, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.
26. A method according to embodiment 25, wherein the mutation is selected from those set out in Table 1 and Table 2.
27. A method according to embodiment 25 or embodiment 26, wherein the haematological malignancy is selected from the group consisting of: Burkitt lymphoma; DLBCL; and ALCL ALK+.
28. A method according to embodiment 20, wherein the gene is selected from the group consisting of: TULP3; PAN3; AC073343.11.1; PHF2; KLHDC1; BRCA2; and WRN, and the analysis identifies elevated expression of the gene.
29. A method of treating a haematological malignancy, the method comprising administering satraplatin to a patient identified as likely to benefit from such treatment on the basis of analysis of a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN.
30. A method according to embodiment 29, wherein the haematological malignancy is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; multiple myeloma; and leukaemia.
31. A method according to embodiment 29 or embodiment 30, wherein the gene is one located in the 9p21 locus, and the analysis identifies a deficiency of the gene.
32. A method according to embodiment 31, wherein the gene is one located in the 9p21 locus, and the analysis identifies reduced copy numbers of the gene.
33. A method according to any of embodiments 29 to 32, wherein the gene is selected from the group consisting of: MTAP; CDKN2B; CDKN2A; and DMRTA1.
34. A method according to any of embodiments 31 to 33, wherein the haematological malignancy is selected from the group consisting of: Burkitt lymphoma; DLBCL; CTCL; multiple myeloma; ALCL ALK+; and mantle cell lymphoma.
35. A method according to embodiment 29, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.
36. A method according to embodiment 39, wherein the mutation is selected from those set out in Table 1 and Table 2.
37. A method according to embodiment 36, wherein the haematological malignancy is selected from the group consisting of: Burkitt lymphoma; DLBCL; and ALCL ALK+.
38. A method according to embodiment 29, wherein the gene is selected from the group consisting of: TULP3; PAN3; AC073343.11.1; PHF2; KLHDC1; BRCA2; and WRN, and the analysis identifies elevated expression of the gene.
39. A method of treating a haematological malignancy, the method comprising: obtaining a sample comprising a cell of the haematological malignancy;
analysing the cell of the haematological malignancy for a change associated with a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; and
administering satraplatin to a patient identified as likely to benefit from such treatment on the basis of the analysis.
40. A method according to embodiment 39, wherein the haematological malignancy is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; multiple myeloma; and leukaemia.
41. A method according to embodiment 39, wherein the gene is one located in the 9p21 locus, and the analysis identifies a deficiency in respect of the gene.
42. A method according to embodiment 41, wherein the deficiency is reduced copy number in respect of the gene.
43. A method according to any of embodiments 39 to 42, wherein the gene is selected from the group consisting of: MTAP; CDKN2B; CDKN2A; and DMRTA1, and the analysis identifies reduced copy numbers of the gene.
44. A method according to embodiment 43, wherein the haematological malignancy is selected from the group consisting of: Burkitt lymphoma; DLBCL; CTCL; multiple myeloma; ALCL ALK+; and mantle cell lymphoma.
45. A method according to embodiment 39, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.
46. A method according to embodiment 39, wherein the mutation is selected from those set out in Table 1 and Table 2.
47. A method according to embodiment 46, wherein the haematological malignancy is selected from the group consisting of: Burkitt lymphoma; DLBCL; and ALCL ALK+.
48. A method according to embodiment 39, wherein the gene is selected from the group consisting of: TULP3; PAN3; AC073343.11.1; PHF2; KLHDC1; BRCA2; and WRN, and the analysis identifies elevated expression of the gene.
49. A method comprising analysing at least one gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; in a cell of a haematological malignancy from a patient, wherein
when the gene is a gene located in the 9p21 locus, the analysis comprises determining if a deficiency is present, for example by assessing copy number;
when the gene is selected from the group consisting of: BCL2; AOC1; BOC; DDX3X; CRYBG3; SLFN5; ETNK2; BRCA2; USP22; KIAA1683; and AP4E1, the analysis comprises assaying for a mutation of the gene; and
when the gene is selected from the group consisting of: TULP3; PAN3; AC073343.11.1; PHF2; KLHDC1; BRCA2; and WRN, the analysis comprises assaying for elevated expression of the gene.
50. A method according to embodiment 49, wherein the haematological malignancy is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; multiple myeloma; and leukaemia.
51. A method according to embodiment 49 or 50, wherein the gene located in the 9p21 locus is selected from the group consisting of: MTAP; CDKN2B; CDKN2A; and DMRTA1.
52. A method according to embodiment 51, wherein the haematological malignancy is selected from the group consisting of: Burkitt lymphoma; DLBCL; CTCL; multiple myeloma; ALCL ALK+; and mantle cell lymphoma.
53. A method according to embodiment 49, wherein the mutation is selected from those set out in Table 1 and Table 2.
54. A method according to embodiment 53, wherein the haematological malignancy is selected from the group consisting of: Burkitt lymphoma; DLBCL; and ALCL ALK+.
55. A method comprising analysing a cell of a haematological malignancy from a patient for the presence of at least one gene mutation selected from the group set out in Table 1 and Table 2.
56. A method according to embodiment 55, wherein the haematological malignancy is selected from the group consisting of: Burkitt lymphoma; DLBCL; and ALCL ALK+.
57. A signature panel characteristic of sensitivity of a haematological malignancy to treatment with satraplatin, the panel comprising:
loss of copy number of at least one gene selected from the group consisting of: MTAP; CDKN2B; CDKN2A; and DMRTA1; and/or
a mutation of at least one gene selected from the group consisting of: BCL2; AOC1; BOC; DDX3X; CRYBG3; SLFN5; ETNK2; BRCA2; USP22; KIAA1683; and AP4E1; and/or
elevated expression of at least one gene selected from the group consisting of: TULP3; PAN3; AC073343.11.1; PHF2; KLHDC1; BRCA2; and WRN.
58. A signature panel according to embodiment 57, wherein the haematological malignancy is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; multiple myeloma; and leukaemia.
59. A signature panel according to embodiment 57, wherein the gene is selected from the group consisting of: MTAP; CDKN2B; CDKN2A; and DMRTA1, and the analysis identifies reduced copy numbers of the gene.
60. A signature panel according to embodiment 59, wherein the haematological malignancy is selected from the group consisting of: Burkitt lymphoma; DLBCL; CTCL; multiple myeloma; ALCL ALK+; and mantle cell lymphoma.
61. A signature panel according to embodiment 57, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.
62. A signature panel according to embodiment 57, wherein the mutation is selected from the group set out in Table 1 and Table 2.
63. A signature panel according to embodiment 62, wherein the haematological malignancy is selected from the group consisting of: Burkitt lymphoma; DLBCL; and ALCL ALK+.
64. A signature panel according to embodiment 57, wherein the gene is selected from the group consisting of: TULP3; PAN3; AC073343.11.1; PHF2; KLHDC1; BRCA2; and WRN, and the analysis identifies elevated expression of the gene.
65. A signature biomarker panel characteristic of sensitivity of a haematological malignancy to treatment with satraplatin, the panel comprising at least one gene mutation selected from the group set out in Table 1 and Table 2.
66. A signature panel according to embodiment 65, wherein the haematological malignancy is selected from the group consisting of: Burkitt lymphoma; DLBCL; and ALCL ALK+.
67. Satraplatin for use as a second line therapy in the treatment of cancer non-responsive to immune checkpoint therapy.

1. Satraplatin for use in the treatment of a haematological malignancy in a patient identified as likely to benefit from such treatment by analysis of a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN.

2. Satraplatin for use according to claim 1, wherein the haematological malignancy is a lymphoma or myeloma.

3. Satraplatin for use according to claim 2, wherein the lymphoma or myeloma is selected from the group consisting of: diffuse large B cell lymphoma (DLBCL); Burkitt lymphoma; mantle cell lymphoma; peripheral T cell lymphoma (PTCL); anaplastic large cell lymphoma (ALCL); primary DLBCL of the central nervous system (CNS); secondary CNS lymphoma (SCNSL); cutaneous T cell lymphoma; Sézary syndrome; and multiple myeloma.

4. Satraplatin for use according to any of claims 1 to 3, wherein the gene is one located in the 9p21 locus, and the analysis identifies reduced copy numbers of the gene.

5. Satraplatin for use according to claim 4, wherein the gene is selected from the group consisting of: MTAP; CDKN2B; CDKN2A; and DMRTA1, and the analysis identifies a deficiency of the gene.

6. Satraplatin for use according to claim 4 or claim 5, wherein the haematological malignancy is selected from the group consisting of: Burkitt lymphoma; DLBCL; CTCL; multiple myeloma; ALCL ALK+; and mantle cell lymphoma.

7. Satraplatin for use according to any of claims 1 to 3, wherein the gene is selected from the group consisting of: BCL2; AOC1; BOC; DDX3X; CRYBG3; SLFN5; ETNK2; BRCA2; USP22; KIAA1683; and AP4E1, and the analysis identifies mutation of the gene.

8. Satraplatin for use according to claim 8, wherein the mutation is selected from those set out in Table 1 and Table 2.

9. Satraplatin for use according to claim 7 or claim 8, wherein the haematological malignancy is selected from the group consisting of: Burkitt lymphoma; DLBCL; and ALCL ALK+.

10. Satraplatin for use according to any of claims 1 to 3, wherein the gene is selected from the group consisting of: TULP3; PAN3; AC073343.11.1; PHF2; KLHDC1; BRCA2; and WRN, and the analysis identifies elevated expression of the gene.

11. Satraplatin for use in the treatment of a haematological malignancy with a loss of the 9p21 locus.

12. Satraplatin for use in the treatment of a haematological malignancy with a deficiency of a gene selected from the group consisting of: MTAP; CDKN2B; CDKN2A; and DMRTA1.

13. Satraplatin for use according to claim 12, wherein the deficiency is reduced copy number of the gene selected from the group consisting of: MTAP; CDKN2B; CDKN2A; and DMRTA1.

14. Satraplatin for use according to any of claims 11 to 13, wherein the haematological malignancy is selected from the group consisting of: Burkitt lymphoma; DLBCL; CTCL; multiple myeloma; ALCL ALK+; and mantle cell lymphoma.

15. Satraplatin for use in the treatment of a haematological malignancy with mutation of a gene selected from the group consisting of: BCL2; AOC1; BOC; DDX3X; CRYBG3; SLFN5; ETNK2; BRCA2; USP22; KIAA1683; and AP4E1.

16. Satraplatin for use according to claim 15, wherein the mutation is selected from the group set out in Table 1 and Table 2.

17. Satraplatin for use according to claim 15 or claim 16, wherein the haematological malignancy is selected from the group consisting of: Burkitt lymphoma; DLBCL; and ALCL ALK+.

18. Satraplatin for use in the treatment of a haematological malignancy with elevated expression of TULP3; PAN3; AC073343.11.1; PHF2; KLHDC1; BRCA2; or WRN.

19. A method of determining the likelihood of a haematological malignancy responding favourably to satraplatin treatment, the method comprising:
analysing a cell of the haematological malignancy for a change in a gene selected from the group consisting of: a gene located in the 9p21 locus (such as MTAP, CDKN2B, CDKN2A, or DMRTA1); BCL2; TULP3; AOC1; BOC; DDX3X; CRYBG3; SLFN5; ETNK2; BRCA2; USP22; KIAA1683; AP4E1; PAN3; AC073343.11.1; PHF2; KLHDC1; and WRN; wherein
identification of a change in the gene analysed indicates that the haematological malignancy is likely to respond favourably to satraplatin treatment.

20. A signature panel characteristic of sensitivity of a haematological malignancy to treatment with satraplatin, the panel comprising:
loss of copy number of at least one gene selected from the group consisting of: MTAP; CDKN2B; CDKN2A; and DMRTA1; and/or
a mutation of at least one gene selected from the group consisting of: BCL2; AOC1; BOC; DDX3X; CRYBG3; SLFN5; ETNK2; BRCA2; USP22; KIAA1683; and AP4E1; and/or
elevated expression of at least one gene selected from the group consisting of: TULP3; PAN3; AC073343.11.1; PHF2; KLHDC1; BRCA2; and WRN.

21. A signature panel according to claim 20, wherein:
the gene with reduced copy number is selected from the group consisting of: MTAP; CDKN2B; CDKN2A; and DMRTA1, and
the haematological malignancy is selected from the group consisting of: Burkitt lymphoma; DLBCL; CTCL; multiple myeloma; ALCL ALK+; and mantle cell lymphoma.

22. A signature panel according to claim 20, wherein:
the gene with a mutation is selected from the group consisting of: BCL2; AOC1; BOC; DDX3X; CRYBG3; SLFN5; ETNK2; BRCA2; USP22; KIAA1683; and AP4E1, and
the mutation is selected from the group set out in Table 1 and Table 2, and
the haematological malignancy is selected from the group consisting of: Burkitt lymphoma; DLBCL; and ALCL ALK+.

23. A signature panel according to claim 20, wherein the gene is selected from the group consisting of: TULP3; PAN3; AC073343.11.1; PHF2; KLHDC1; BRCA2; and WRN, and the analysis identifies elevated expression of the gene.

24. A signature biomarker panel characteristic of sensitivity of a haematological malignancy to treatment with satraplatin, the panel comprising at least one gene mutation selected from the group set out in Table 1 and Table 2, wherein the haematological malignancy is selected from the group consisting of: Burkitt lymphoma; DLBCL; and ALCL ALK+.

25. Satraplatin for use as a second line therapy in the treatment of cancer non-responsive to immune checkpoint therapy.
